(19) United States
(12) Patent Application Publication
(10) Pub. No.: US 2025/0304931 A1
(43) Pub. Date: Oct. 2, 2025

(54) NOVEL MUTATED LACTONASE ENZYMES, COMPOSITIONS CONTAINING THEM AND USES THEREOF

(71) Applicants: GENE AND GREEN TK, MARSEILLE (FR); FONDATION MEDITERRANEE INFECTION, MARSEILLE (FR); AIX-MARSEILLE UNIVERSITE, MARSEILLE CEDEX 07 (FR); REGENTS OF THE UNIVERSITY OF MINNESOTA, Minneapolis, MN (US)

(72) Inventors: David DAUDE, MARSEILLE (FR); Mikael ELIAS, Centerville, MN (US); Eric CHABRIERE, MARSEILLE (FR); Laure PLENER, MARSEILLE (FR); Raphaël BILLOT, MARSEILLE (FR)

(73) Assignees: GENE AND GREEN TK, MARSEILLE (FR); FONDATION MEDITERRANEE INFECTION, MARSEILLE (FR); AIX-MARSEILLE UNIVERSITE, MARSEILLE CEDEX 07 (FR); REGENTS OF THE UNIVERSITY OF MINNESOTA, Minneapolis, MN (US)

(21) Appl. No.: 18/839,183

(22) PCT Filed: Feb. 15, 2023
(86) PCT No.: PCT/EP2023/053717

§ 371 Date: Aug. 16, 2024

(30) Foreign Application Priority Data
Feb. 17, 2022 (FR) FR2201424

(51) Int. Cl. C12N 9/18; A01N 63/50; A01P 1/00; A61K 38/00

(52) U.S. Cl. CPC C12N 9/18; A01N 63/50; C12Y 301/01081; A01P 1/00; A61K 38/00

(57) ABSTRACT

The present invention relates to new mutated lactonases, to their uses as well as to compositions containing them.

### REFERENCE TO A SEQUENCE LISTING

In accordance with 37 CFR § 1.831, the present specification makes reference to a Sequence Listing submitted electronically as a .xml file named “Substitute_Sequence_Listing”. The .xml file was generated on Mar. 7, 2025 and is 67,482 bytes in size. The entire contents of the Sequence Listing are hereby incorporated by reference.

### FIELD

The present invention concerns new mutated lactonases, their uses and compositions containing them.

### BACKGROUND

Some bacteria use a molecular communication system called Quorum Sensing (QS) to coordinate numerous biological functions such as virulence or biofilm formation. In particular, they use acyl-homoserine lactones (AHL) as communication molecules.

Enzymes in the phosphotriesterase-like lactonase (PLL) family have both phosphotriesterase activity and lactonase activity and are capable of hydrolysing the homoserine lactones, involved in bacterial SQ, with varying degrees of efficiency.

Bacterial biofilm formation causes both medical and environmental problems, so it's important to find solutions for effectively eliminating bacterial biofilms.

### SUMMARY

In a first aspect, the invention concerns new mutated lactonases.

In a second aspect, the invention concerns the use of said mutated lactonases.

In a third aspect, the invention concerns compositions comprising said mutated lactonases.

In a fourth aspect, the invention concerns a method for preventing and/or treating pathologies linked to bacterial infections.

Surprisingly, the inventors of the present application have shown that one or more mutations in the sequence of the lactonase enzymes significantly increase the efficiency of the lactonases towards the AHLs involved in bacterial QS.

In all aspects of the present invention, the said mutated lactonase enzyme is derived from the hyperthermophilic lactonase of Saccharolobus solfataricus (SsoPox), Sulfolobus acidocalaricus, Sulfolobus islandicus or Saccharolobus shibatae belonging to the phosphotriesterase-like lactonase family.

### BRIEF DESCRIPTION OF THE FIGURES

FIG. 1—Relative lactonase activities of loop 8 alanine-scanning SsoPox variants using the reporter strains CV026 and Pseudomonas putida KS35 (A) and (B): The production of violacein by CV026, induced by the addition of C4-HSL and C6-HSL, was measured in greyscale with ImageJ software and transformed into relative activity. A high relative activity corresponds to the absence of violacein production by the reporter strain. (C), (D) and (E): The fluorescence emitted by P. putida KS35 in the presence of 3-oxo-C8-HSL, 3-oxo-C10-HSL and 3-oxo-C12-HSL was measured and transformed into relative activity. A high relative activity corresponds to low fluorescence emission from the reporter strain. SsoPox V82I is the base enzyme for mutagenesis.

FIG. 2—General representation of the activities of SsoPox variants mutated at loop 8. The logarithmic values of kcat/K_{M} (s⁻¹·M⁻¹) are displayed on the circles, and are also represented by shades of grey ranging from white to black. The improvement in activity compared with SsoPox WT is represented by the size of the circles.

FIG. 3—Progressive decrease in violacein produced by Chromobacterium violaceum treated with SsoPox V82I and SsoPox V82I/A275G after 16 hours growth at 30° C. Dots represent bacterial growth measured by 600 nm absorbance. Bars represent extracted violacein measured by absorbance 585 nm.

FIG. 4—Bioluminescence of Vibrio harveyi treated with SsoPox 5A8 (inactive), SsoPox V82I and SsoPox V82I/A275G, after 24 hours growth at 30° C.

FIG. 5—Growth and prodigiosin production of Serratia sp. 39006 untreated or treated with SsoPox 5A8 (inactive), SsoPox V82I and SsoPox V82I/A275G at 0.25 mg·mL⁻¹. Bacteria were grown for 24 hours at 30° C. The black bars represent bacterial growth measured by absorbance at 600 nm and the grey bars represent prodigiosin production measured by absorbance at 534 nm after extraction.

FIG. 6—Number of peptides linked to carbapenem production identified in a 24-hour culture of Serratia sp. 39006, untreated or treated with SsoPox V82I and SsoPox V82I/A275G at 0.25 mg·mL⁻¹.

FIG. 7—A: Total number of peptides identified, by size, in the 3 Serratia sp. 39006 culture conditions. B: Variation in the number of peptides detected compared with the untreated control.

FIG. 8—Principal component analyses on 506 proteins from Serratia sp. 39006. Proteins selected have at least 2 peptides enumerated and a normalised spectral abundance factor ≥0.05%.

FIG. 9—The SsoPox V82I variant inhibits infection by P. atrosepticum CFBP6276 in potatoes.

FIG. 10—Alignment of PLL sequences from extremophilic archaea. A: SisLac vs SsoPox alignment. B: SacPox vs SsoPox alignment. C: Multiple alignment (SsoPox, SisLac and SacPox) with Clustal Omega. The top bar represents the variation in the consensus sequence, from black (same residue) to light grey (completely different residue). The black bar indicates loop 8.

FIG. 11—Alignment of extremophilic archaeal PLL sequences using Clustal Omega. The vertical bars indicate the frequency of an amino acid in all the aligned sequences, from black (same residue in all the sequences) to light grey (several different residues). The consensus sequence is defined with a threshold of 70%. The light grey horizontal bar indicates loop 8 and the black horizontal bar indicates zone L242-P289.

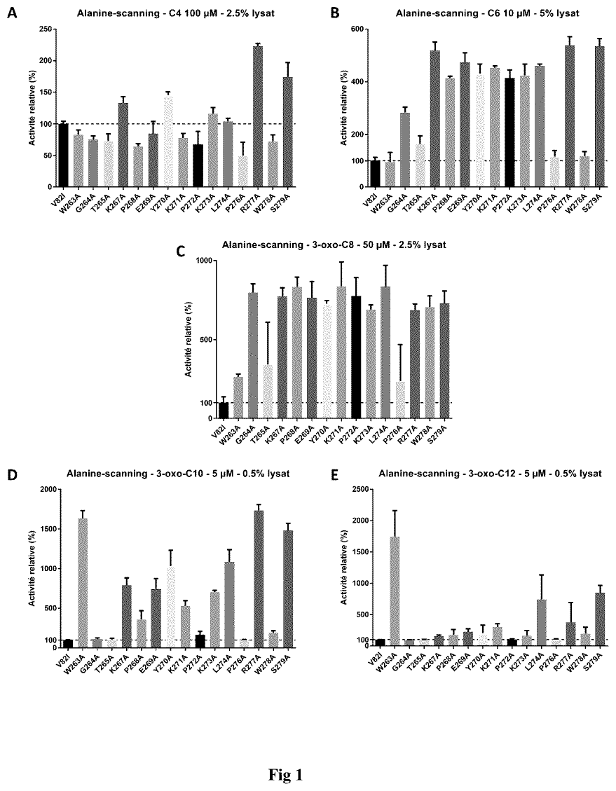

### DETAILED DESCRIPTION

In a first aspect, the invention concerns new mutated lactonases.

In particular, the inventors of the present application have identified that the mutation of an amino acid X in the consensus sequence of a wild-type lactonase consisting of: I-R-F-[M/S]-E-[K/R]-X-V-K-[A/T/E]-T-G-I-N (SEQ ID NO: 1) and that at least one mutation of an amino acid Xₐ-Xⱼ of loop 8 of phosphotriesterase-like lactonases consisting of: Xa-G-[T/I]-Xb-[K/R]-P-E-Xc-Xd-Xe-Xf-Xg-Xh-P-Xi-W-Xj (SEQ ID NO: 3), made it possible to obtain a mutated lactonase with increased hydrolysis activity on homoserine lactone substrates compared with the said wild-type lactonase.

In the present invention, the expression “increased lactonase hydrolysis activity” means that, for the hydrolysis of a homoserine lactone substrate, the lactonase mutated according to the invention has a higher Kcat/K_{M} ratio value compared with the Kcat/K_{M} ratio value of the non-mutated lactone from which it is derived.

To obtain kcat and K_{M} values, the enzymatic hydrolysis of lactones was monitored over time. The opening of the lactone ring by hydrolysis leads to the release of an acid function, so the parameters were determined by monitoring the acidification of the reaction medium. The measurement was carried out in buffer (2.5 mM bicin pH 8.3, 150 mM NaCl, 0.2 mM CoCl₂, 0.25 mM cresol violet and 0.5% DMSO). Cresol violet is a pH indicator used to monitor the acidification of the medium caused by hydrolysis of the lactone ring. Hydrolysis was monitored by measuring the change in absorbance of the reaction medium at λ=577 nm for 10 minutes. Each point was performed in triplicate and Gen5.1 software was used to evaluate the initial degradation rate at each substrate concentration. The kcat and K_{M} values were obtained using a regression of the Michaelis-Menten equation with GraphPad Prism 7 software.

Thus, in a first embodiment, the invention relates to a mutated lactonase belonging to the family of hyperthermophilic phosphotriesterase-like lactonases, said mutated lactonase comprising
a first mutation by substitution of an amino acid in a first consensus sequence SEQ ID NO: 1 of a wild-type lactonase,
   which first consensus sequence in said wild-type lactonase is represented by SEQ ID: 1:

(SEQ ID: 1)
I-R-F-[M/S]-E-[K/R]-X-V-K-[A/T/E]-T-G-I-N

X represents the V amino acid,
which first consensus sequence in said mutated lactonase is represented by SEQ ID NO: 2:

(SEQ ID NO: 2)
I-R-F-[M/S]-E-[K/R]-X1-V-K-[A/T/E]-T-G-I-N

X₁ represents the substituted amino acid chosen from the group consisting of the hydrophobic amino acids V, I, L, M, F, G, A, P, W, Y and C, in particular A, G and I, in particular A or I,
at least one other mutation by substitution of an amino acid in a second consensus sequence of the wild-type lactonase represented by SEQ ID: 3:

(SEQ ID NO: 3)
Xa-G-[T/I]-Xb-[K/R]-P-E-Xc-Xd-Xe-Xf-Xg-Xh-P-Xi-
W-Xj,

Xa is selected from the group consisting of W, T, A, F, V, I, M and L,
Xb represents the amino acid A,
Xc is selected from the group consisting of Y and L,
Xd represents the amino acid K,
Xe represents the amino acid P,
Xf represents the amino acid K,
Xg represents the amino acid L,
Xh represents the amino acid A,
Xi is selected from the group consisting of R and K,
Xj represents the amino acid S,
which second consensus sequence in said substitution-mutated lactonase is represented by SEQ ID: 4:

(SEQ ID NO: 4)
X₂-G-[T/I]-X₃-[K/R]-P-E-X₄-X₅-X₆-X₇-X₈-X₉-W-X₁₀-P-X₁₁

at least one of the amino acids X₂, X₃, X₄, X₅, X₆, X₇, X₈, X₉, X₁₀ or X₁₁ being substituted,
said mutated lactonase having increased lactonase activity compared with said wild-type lactonase, preferably on at least one substrate.

The Inventors of the present application have also shown that these mutations make it possible to obtain a mutated lactonase with greatly improved hydrolysis activity on lactone homoserine substrates compared with the said wild-type lactonase, making it possible to change the specificity spectrum of lactonases and/or to increase activity towards lactone homoserine substrates.

In a particular embodiment, the invention relates to a mutated lactonase as described above exhibiting increased lactonase activity compared with said wild-type lactonase on at least one substrate selected from: C4-HSL, C6-HSL, 3-oxo-C6-HSL, C8-HSL, 3-oxo-C10-HSL, 3-oxo-C12-HSL and 3-oxo-C8-HSL. These substrates are homoserine lactones and are found in quorum sensing, enabling bacteria to communicate.

The expression “greatly enhanced hydrolysis activity” here means that mutated lactonase as defined above has up to 297 times greater hydrolysis activity on homoserine lactone substrates than said wild-type lactonase.

In another embodiment, the invention relates to a mutated lactonase belonging to the family of hyperthermophilic phosphotriesterase-like lactonases, said mutated lactonase comprising
a first mutation by substitution of an amino acid in a first consensus sequence SEQ ID NO: 1 of a wild-type lactonase,
   which first consensus sequence in said wild-type lactonase is represented by SEQ ID: 1:

(SEQ ID: 1)
I-R-F-[M/S]-E-[K/R]-X-V-K-[A/T/E]-T-G-I-N

X represents the V amino acid,
which first consensus sequence in said mutated lactonase is represented by SEQ ID NO: 2:

(SEQ ID NO: 2)
I-R-F-[M/S]-E-[K/R]-X1-V-K-[A/T/E]-T-G-I-N

X₁ represents the substituted amino acid chosen from the group consisting of the hydrophobic amino acids V, I, L, M, F, G, A, P, W, Y and C, in particular A, G and I, in particular A or I,
at least one other mutation by substitution of an amino acid in a second consensus sequence of the wild-type lactonase represented by SEQ ID: 3:

(SEQ ID NO: 3)
Xa-G-[T/I]-Xb-[K/R]-P-E-Xc-Xd-Xe-Xf-Xg-Xh-P-Xi-
W-Xj,

Xa is selected from the group consisting of W, T, A, F, V, I, M and L,
Xb represents the A amino acid,
Xc is selected from the group consisting of Y and L,
Xd represents the K amino acid,
Xe represents the P amino acid,
Xf represents the K amino acid,
Xg represents the L amino acid,
Xh represents the A amino acid,
Xi is selected from the group consisting of R and K,
Xj represents the S amino acid,
which second consensus sequence in said substitution-mutated lactonase is represented by SEQ ID:4:

(SEQ ID NO: 4)
X2-G-[T/I]-X₃-[K/R]-P-E-X₄-X₅-X₆-X₇-X₈-X₉-W-X1₀-P-X₁₁

at least one of the amino acids X₂, X₃, X₄, X₅, X₆, X₇, X₈, X₉, X₁₀ or X₁₁ being substituted,
X₂ is selected from the group consisting of the hydrophobic amino acids V, I, L, M, F, G, A, P, W, Y and C, in particular A, G and I, in particular A,
X₃ is selected from the group consisting of the hydrophobic amino acids V, I, L, M, F, G, P, W, Y and C, in particular G, I, M and V, in particular G,
X₄ is selected from the group consisting of the hydrophobic amino acids V, I, L, M, F, G, A, P, W, Y and C, in particular F,
X₅ is selected from the group consisting of the hydrophobic amino acids V, I, L, M, F, G, A, P, W, Y and C, in particular L,
X₆ is selected from the group consisting of the hydrophobic amino acids V, I, L, M, F, G, A, W, Y and C, in particular L,
X₇ is selected from the group consisting of the polar amino acids S, T, N, Q, E, D, R and H, in particular N,
X₈ is selected from the group consisting of the hydrophobic amino acids V, I, M, F, G, A, P, W, Y and C, in particular V,
X₉ is selected from the group consisting of the hydrophobic amino acids V, I, L, M, F, G, P, W, Y and C, in particular G, M and W,
X₁₀ is selected from the group consisting of the non-voluminous amino acids G, P, L, I, A, D, C, S, T and N, in particular A,
X₁₁ is selected from the group consisting of the hydrophobic amino acids V, I, L, M, F, G, A, P, W, Y and C, in particular A,
   said mutated lactonase having increased lactonase activity compared with said wild-type lactonase, preferably on at least one substrate.

In a particular embodiment, X₁ is the amino acid Isoleucine I.

In a particular embodiment, the invention relates to a mutated lactonase as described above, wherein X₁ is the I amino acid and said substrate is selected from: C4-HSL, C6-HSL, 3-oxo-C6-HSL, C8-HSL and 3-oxo-C12-HSL.

In another embodiment, the invention relates to a mutated lactonase belonging to the family of hyperthermophilic phosphotriesterase-like lactonases, said mutated lactonase comprising
a first mutation by substitution of an amino acid in a first consensus sequence SEQ ID NO: 1 of a wild-type lactonase,
   which first consensus sequence in said wild-type lactonase is represented by SEQ ID: 1:

(SEQ ID: 1)
I-R-F-[M/S]-E-[K/R]-X-V-K-[A/T/E]-T-G-I-N

X represents the V amino acid,
which first consensus sequence in said mutated lactonase is represented by SEQ ID NO: 2:

(SEQ ID NO: 2)
I-R-F-[M/S]-E-[K/R]-X1-V-K-[A/T/E]-T-G-I-N

X₁ represents the substituted amino acid chosen from the group consisting of the hydrophobic amino acids V, I, L, M, F, G, A, P, W, Y and C, in particular A, G and I, in particular A or I,
at least one other mutation by substitution of an amino acid in a second consensus sequence of the wild-type lactonase represented by SEQ ID: 3:

(SEQ ID NO: 3)
Xa-G-[T/I]-Xb-[K/R]-P-E-Xc-Xd-Xe-Xf-Xg-Xh-P-Xi-
W-Xj,

Xa is selected from the group consisting of W, T, A, F, V, I, M and L,
Xb represents the amino acid A,
Xc is selected from the group consisting of Y and L,
Xd represents the amino acid K,
Xe represents the amino acid P,
Xf represents the amino acid K,
Xg represents the amino acid L,
Xh represents the amino acid A,
Xi is selected from the group consisting of R and K,
Xj represents the amino acid S,
which second consensus sequence in said substitution-mutated lactonase is represented by SEQ ID:4:

(SEQ ID NO: 4)
X2-G-[T/I]-X₃-[K/R]-P-E-X₄-X₅-X₆-X₇-X₈-X₉-W-X1₀-P-X₁₁

at least one of the amino acids X₂, X₃, X₄, X₅, X₆, X₇, X₈, X₉, X₁₀ or X₁₁ being substituted,
X₂ is selected from the group consisting of A and I,
X₃ is selected from the group consisting of V, I, M, G and T,
X₄ represents F,
X₅ represents L,
X₆ represents L,
X₇ represents N,
X₈ represents V,
X₉ is selected from the group consisting of F, M, G, Y, C, and W,
X₁₀ represents A,
X₁₁ represents A.
said mutated lactonase having increased lactonase activity compared with said wild-type lactonase, preferably on at least one substrate.

In a particular embodiment, the at least one other mutation by substitution of an amino acid in the consensus sequence of the wild-type lactonase represented by SEQ ID: 3 concerns amino acid X₂ of the sequence SEQ ID: 4 of the mutated lactonase,
X₂ is selected from the group consisting of the hydrophobic amino acids V, I, L, M, F, G, A, P, W, Y and C, in particular A, G and I, especially A.

In a particular embodiment, the at least one other mutation by substitution of an amino acid in the consensus sequence of the wild-type lactonase represented by SEQ ID: 3 concerns amino acid X₃ of the sequence SEQ ID: 4 of the mutated lactonase,
X₃ is selected from the group consisting of the hydrophobic amino acids V, I, L, M, F, G, P, W, Y and C, in particular G, I, M, T and V, in particular G.

In a particular embodiment, the at least one other mutation by substitution of an amino acid in the consensus sequence of the wild-type lactonase represented by SEQ ID: 3 concerns amino acid X₄ of the sequence SEQ ID: 4 of the mutated lactonase,
X₄ is selected from the group consisting of the hydrophobic amino acids V, I, L, M, F, G, A, P, W, Y and C, in particular F.

In a particular embodiment, the at least one other mutation by substitution of an amino acid in the consensus sequence of the wild-type lactonase represented by SEQ ID: 3 concerns amino acid X₅ of the sequence SEQ ID: 4 of the mutated lactonase,
X₅ is selected from the group consisting of the hydrophobic amino acids V, I, L, M, F, G, A, P, W, Y and C, in particular L.

In a particular embodiment, the at least one other mutation by substitution of an amino acid in the consensus sequence of the wild-type lactonase represented by SEQ ID: 3 concerns amino acid X₆ of the sequence SEQ ID: 4 of the mutated lactonase,
X₆ is selected from the group consisting of the hydrophobic amino acids V, I, L, M, F, G, A, W, Y and C, in particular L.

In a particular embodiment, the at least one other mutation by substitution of an amino acid in the consensus sequence of the wild-type lactonase represented by SEQ ID: 3 concerns amino acid X₇ of the sequence SEQ ID: 4 of the mutated lactonase,
X₇ is selected from the group consisting of the polar amino acids S, T, N, Q, E, D, R and H, in particular N.

In a particular embodiment, the at least one other mutation by substitution of an amino acid in the consensus sequence of the wild-type lactonase represented by SEQ ID: 3 concerns amino acid X₈ of the sequence SEQ ID: 4 of the mutated lactonase,
X₈ is selected from the group consisting of the hydrophobic amino acids V, I, M, F, G, A, P, W, Y and C, in particular V.

In a particular embodiment, the at least one other mutation by substitution of an amino acid in the consensus sequence of the wild-type lactonase represented by SEQ ID: 3 concerns amino acid X₉ of the sequence SEQ ID: 4 of the mutated lactonase,
X₉ is selected from the group consisting of the hydrophobic amino acids V, I, L, M, F, G, P, W, Y and C, in particular G, M and W.

In a particular embodiment, the at least one other mutation by substitution of an amino acid in the consensus sequence of the wild-type lactonase represented by SEQ ID: 3 concerns amino acid X₁₀ of the sequence SEQ ID: 4 of the mutated lactonase,
X₁₀ is selected from the group consisting of the non-voluminous amino acids G, P, L, I, A, D, C, S, T and N, in particular A.

In a particular embodiment, the at least one other mutation by substitution of an amino acid in the consensus sequence of the wild-type lactonase represented by SEQ ID: 3 concerns amino acid X₁₁ of the sequence SEQ ID: 4 of the mutated lactonase,
X₁ is selected from the group consisting of the hydrophobic amino acids V, I, L, M, F, G, A, P, W, Y and C, in particular A.

In another embodiment, the invention relates to a mutated lactonase belonging to the family of hyperthermophilic phosphotriesterase-like lactonases, said mutated lactonase comprising
a first mutation by substitution of an amino acid in a first consensus sequence SEQ ID NO: 1 of a wild-type lactonase,
   which first consensus sequence in said wild-type lactonase is represented by SEQ ID: 1:

(SEQ ID: 1)
I-R-F-[M/S]-E-[K/R]-X-V-K-[A/T/E]-T-G-I-N

X represents the amino acid,
which first consensus sequence in said mutated lactonase is represented by SEQ ID NO: 2:

(SEQ ID NO: 2)
I-R-F-[M/S]-E-[K/R]-X1-V-K-[A/T/E]-T-G-I-N

X₁ represents the I amino acid,
at least one other mutation by substitution of an amino acid in a second consensus sequence of the wild-type lactonase represented by SEQ ID: 3:

(SEQ ID NO: 3)
Xa-G-[T/I]-Xb-[K/R]-P-E-Xc-Xd-Xe-Xf-Xg-Xh-P-Xi-
W-Xj,

Xa is selected from the group consisting of W, T, A, F, V, I, M and L,
Xb represents the A amino acid,
Xc is selected from the group consisting of Y and L,
Xd represents the K amino acid,
Xe represents the P amino acid,
Xf represents K the amino acid,
Xg represents L the amino acid,
Xh represents A the amino acid,
Xi is selected from the group consisting of R and K,
Xj represents the S amino acid,
which second consensus sequence in said substitution-mutated lactonase is represented by SEQ ID:4:

(SEQ ID NO: 4)
X2-G-[T/I]-X₃-[K/R]-P-E-X₄-X₅-X₆-X₇-X₈-X₉-W-X1₀-P-X₁₁

at least one of the amino acids X₂, X₃, X₄, X₅, X₆, X₇, X₈, X₉, X₁₀ or X₁₁ being substituted,
X₂ is selected from the group consisting of A and I,
X₃ is selected from the group consisting of V, I, M, G and T,
X₄ represents F,
X₅ represents L,
X₆ represents L,
X₇ represents N,
X₈ represents V,
X₉ is selected from the group consisting of F, M, G, Y, C and W,
X₁₀ represents A,
X₁₁ represents A.
said mutated lactonase having increased lactonase activity compared with said wild-type lactonase, preferably on at least one substrate.

In another preferred embodiment, the invention concerns a mutated lactonase belonging to the family of hyperthermophilic phosphotriesterase-like lactonases represented by the sequence SEQ ID NO: 4 in which a single mutation by substitution concerns one of the amino acids chosen from the group consisting of X₂, X₃, X₄, X₅, X₆, X₇, X₈, X₉, X₁₀ and X₁₁.

In another preferred embodiment, the invention concerns a mutated lactonase belonging to the family of hyperthermophilic phosphotriesterase-like lactonases represented by the sequence SEQ ID NO: 4 in which at least two mutations by substitution concern at least two of the amino acids chosen from the group consisting of X₂, X₃, X₄, X₅, X₆, X₇, X₈, X₉, X₁₀ and X₁₁.

In another embodiment, the invention relates to a mutated lactonase belonging to the family of hyperthermophilic phosphotriesterase-like lactonases in which the at least one other mutation by substitution relates to the amino acids X₃, and X₉, of the sequence SEQ ID: 4 in which: X₃ is selected from the group consisting of I and G, X₉ is selected from the group consisting of F, M, Y and C.

Table 1 below summarizes the particularly preferred mutations described in this second embodiment of this first aspect. These mutations are carried out in the second consensus sequence (SEQ ID NO: 3) of the wild-type lactonase and make it possible to obtain the mutated lactonases described in this second embodiment of this first aspect.

| | X [symbol] / | | | X [symbol] / | | | | X [symbol] / | X [symbol] / | X [symbol] / | X [symbol] / | X [symbol] / | X [symbol] / | | X [symbol] / | | X [symbol] / |
|---|---|---|---|---|---|---|---|---|---|---|---|---|---|---|---|---|---|
| | X [symbol] | | | X [symbol] | | | | X [symbol] | X [symbol] | X [symbol] | X [symbol] | X [symbol] | X [symbol] | | X [symbol] | | X [symbol] |
| SECOND | [symbol] | α | [symbol] | A | [symbol] | [symbol] | E | [symbol] | K | ρ | [symbol] | [symbol] | A | ρ | [symbol] | W | [symbol] |
| CONSENSUS | | | | | | | | | | | | | | | | | |
| SEQUENCE | | | | | | | | | | | | | | | | | |
| WT (SEQ ID | | | | | | | | | | | | | | | | | |
| NO: 3) | | | | | | | | | | | | | | | | | |
| SUMMARY | A | | | | | | | | | | | | | | | | |
| OF THE | [symbol] | | | | | | | | | | | | | | | | |
| MUTATIONS | V | | | | | | | | | | | | | | | | |
| IN THE | T | | | | | | | | | | | | | | | | |
| SECOND | | | | G | | | | | | | | | | | | | |
| CONSENSUS | | | | [symbol] | | | | | | | | | | | | | |
| SEQUENCE | | | | M | | | | | | | | | | | | | |
| (SEQ ID NO: 4) | | | | T | | | | | | | | | | | | | |
| TO OBTAIN | | | | Y | | | | | | | | | | | | | |
| THE MUTATED | | | | | | | | [symbol] | | | | | | | | | |
| LACTONASES | | | | | | | | | L | | | | | | | | |
| OF THE | | | | | | | | | | [symbol] | | | | | | | |
| INVENTION | | | | | | | | | | | N | | | | | | |
| | | | | | | | | | | | | [symbol] | | | | | |
| | | | | | | | | | | | | | G | | | | |
| | | | | | | | | | | | | | M | | | | |
| | | | | | | | | | | | | | W | | | | |
| | | | | | | | | | | | | | | | A | | |
| | | | | | | | | | | | | | | | | | A |
| | | | | G | | | | | | | | | [symbol] | | | | |
| | | | | G | | | | | | | | | M | | | | |
| | | | | G | | | | | | | | | Y | | | | |
| | | | | [symbol] | | | | | | | | | [symbol] | | | | |

[symbol]

indicates data missing or illegible when filed

In another embodiment, the invention relates to a mutated lactonase belonging to the family of hyperthermophilic phosphotriesterase-like lactonases in which the said mutated lactonase has a sequence identity of at least 85%, 90%, 95%, 96%, 97%, 98%, 99% or 100% with the sequences SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47, provided that said mutated lactonase retains increased lactonase activity relative to said wild-type lactonase.

In this particular embodiment, it is understood that any variation in the sequence of the said mutated lactonase leading to a sequence identity of at least 85%, 90%, 95%, 96%, 97%, 98%, 99% or 100% with the sequences SEQ ID NO: 26 to SEQ ID NO: 47 concerns all the positions other than those of amino acid X in the consensus sequence of a wild-type lactonase consisting of: I-R-F-[M/S]-E-[K/R]-X-V-K-[A/T/E]-T-G-I-N (SEQ ID NO: 1) and amino acids Xa-Xj of loop 8 of phosphotriesterase-like lactonases consisting of: Xa-G-[T/I]-Xb-[K/R]-P-E-Xc-Xd-Xe-Xf-Xg-Xh-P-Xi-W-Xj (SEQ ID NO: 3). For example, a mutated lactonase which has 98% identity with the sequence SEQ ID NO: 26, which corresponds to the SsoPox V82I/W263A mutant, may have 1 to 6 other mutations from 303 possible positions out of 314 amino acids of the sequence SEQ ID NO: 26, it being understood that the positions X and Xa-Xj of the consensus sequences SEQ ID NO: 1 and SEQ ID NO: 3 must correspond to the various embodiments of the present invention.

In any of the previously described embodiments, said substrate may be selected from: C4-HSL, C6-HSL, 3-oxo-C6-HSL, C8-HSL, 3-oxo-C10-HSL, 3-oxo-C12-HSL and 3-oxo-C8-HSL. These substrates are homoserine lactones found in quorum sensing, enabling bacteria to communicate.

In all aspects of the present invention, said wild-type lactonase may be selected from Saccharolobus solfataricus (SsoPox), Sulfolobus acidocalaricus, Sulfolobus islandicus and Saccharolobus shibatae.

In all aspects of the present invention, and according to a particular embodiment of this first aspect, said wild-type lactonase is chosen from Saccharolobus solfataricus (SsoPox) of sequence SEQ ID NO: 5, Sulfolobus acidocalaricus of sequence SEQ ID NO: 6, Sulfolobus islandicus of sequence SEQ ID NO: 7 and Saccharolobus shibatae of sequence SEQ ID NO: 8.

In all aspects of the present invention, said mutated lactonase has a lactonase activity increased by at least 2-fold, preferably from 2 to 70-fold, more preferably from 40 to 50-fold, compared with said wild-type lactonase, on at least one substrate.

In the present invention, the expression “increased lactonase hydrolysis activity” means that, for the hydrolysis of a homoserine lactone substrate, the lactonase mutated according to the invention has a higher Kcat/K_{M} ratio value compared with the Kcat/K_{M} ratio value of the non-mutated lactone from which it is derived.

Thus, this means that the Kcat/K_{M} of the mutated lactonase according to the invention is increased by at least two times, preferably between 25 and 70 times and more preferably 40 to 50 times, compared with the non-mutated lactonase.

In another embodiment, the invention concerns the use of a mutated lactonase belonging to the family of hyperthermophilic phosphotriesterase-like lactonases, as described above, said mutated lactonase having increased lactonase activity compared with said wild-type lactonase, in particular on at least one substrate, for:
disrupting the quorum-sensing of bacteria using homoserine lactone substrates to communicate,
limiting or inhibiting the formation of biofilms.

In the present invention, the term “bacteria” refers to a genus of prokaryotic microorganisms scientifically classified as such. Most bacteria can be classified as Gram-positive or Gram-negative.

In a particular embodiment, the bacteria can be selected from gram-positive and gram-negative bacteria.

According to the present invention, “Gram-positive bacteria” are bacteria bound by a single lipid membrane and containing a thick layer of peptidoglycans (20 to 80 nm) that retain crystal violet staining in a Gram staining technique.

According to the present invention, “Gram-negative bacteria” are bacteria bound by a cytoplasmic membrane as well as by an outer cell membrane, containing only a thin layer of peptidoglycans between the two membranes, which does not allow the crystal violet dye to be retained in a Gram staining technique.

More particularly, said bacteria may be selected from the group consisting of: Aeromonas sp., Aliivibrio sp., Edwardsiella sp., Enterobacter sp., Halomonas sp., Pantoea sp., Pseudomonas sp., Serratia sp., Vibrio sp., Acinetobacter sp., Agrobacterium sp., Azospirillum sp., Burkholderia sp., Chromobacterium sp., Dickeya sp., Erwinia sp., Hafnia sp. Klebsiella sp., Methylobacterium sp., Pectobacterium sp., Ralstonia sp., Rhizobium sp., Sinorhizobium sp., Yersinia sp., Castellaniella sp., Dinoroseobacter sp., Gluconacetobacter sp., Mesorhizobium sp., Pandoraea sp., Proteus sp., Roseobacter sp., Nitrobacter sp., Rhodospirillum sp., Acidithiobacillus sp., Brucella sp., Kluyvera sp., Photobacterium sp., Rahnella sp. and Brayrhizobium sp.

In another particular embodiment, the invention relates to a composition comprising as active principle at least one mutated lactonase as defined above.

In a particular embodiment, said composition may also contain a pharmaceutically acceptable vehicle.

In a particular embodiment, said composition described above may be formulated with at least one suitable excipient for use as a solution, oil, suspension, emulsion, nanoparticles, liposomes, granules or functionalized surface.

Thus, in this particular embodiment, the invention relates to an antibacterial composition comprising as active principle at least one mutated lactonase as defined above.

In a particular embodiment, said composition may also contain a pharmaceutically acceptable vehicle.

In a particular embodiment, said composition described above may be formulated with at least one suitable excipient for use as a solution, oil, suspension, emulsion, nanoparticles, liposomes, granules or functionalized surface.

Thus, in this particular embodiment, the invention relates to a phytosanitary composition comprising as active ingredient at least one mutated lactonase as defined above.

In this embodiment, the invention also relates to a composition comprising as active ingredient at least one mutated lactonase as defined above for use in human health, in particular for the prevention and/or treatment of pathologies linked to bacterial infections.

In all aspects of the present invention, by “treatment” it means the means of curing a declared pathology, the symptoms of which are visible. In all aspects of the present invention, “prevention” means the means of preventing the said pathology from occurring.

In a particular embodiment, said bacterial infections are caused by bacteria using homoserine lactone substrates to communicate.

In this embodiment, the invention also relates to a composition comprising as active ingredient at least one mutated lactonase as defined above for use in human health, in particular for the prevention and/or treatment of bacterial infections, such as pneumonia or nosocomial diseases, wounds, burns, eye infections, diabetic foot, for the prevention and/or treatment of dysbiosis, or for the prevention and/or treatment of dental plaque.

In a particular embodiment, said bacteria are in particular selected from: Acinetobacter sp., Brucella sp., Burkholderia sp., Chromobacterium sp., Enterobacter sp., Hafnia sp., Klebsiella sp., Kluyvera sp., Pandoraea sp., Proteus sp., Pseudomonas sp., Rahnella sp., Vibrio sp. and Yersinia sp.

In a particular embodiment, said composition may also contain a pharmaceutically acceptable vehicle.

In a particular embodiment, said composition described above may be formulated with at least one suitable excipient for use as a solution, oil, suspension, emulsion, nanoparticles, liposomes, granules or functionalized surface.

In this particular embodiment, the invention also relates to a composition comprising as active ingredient at least one mutated lactonase as defined above for use in animal health, in particular for the prevention and/or treatment of bacterial infections, the prevention and/or treatment of dysbiosis, and the prevention and/or elimination of biofilms present in breeding tanks and aquaria.

In a particular embodiment, said bacterial infections are caused by bacteria using homoserine lactone substrates to communicate.

In a particular embodiment, said bacteria are notably selected from: Aeromonas sp., Aliivibrio sp., Brucella sp., Burkholderia sp., Chromobacterium sp., Edwardsiella sp., Enterobacter sp., Halomonas sp., Pseudomonas sp., Vibrio sp. and Yersinia sp.

In a particular embodiment, said composition may also contain a pharmaceutically acceptable vehicle.

In a particular embodiment, said composition described above may be formulated with at least one suitable excipient for use as a solution, oil, suspension, emulsion, nanoparticles, liposomes, granules or functionalized surface.

In another particular embodiment, the invention also relates to a composition comprising as active ingredient at least one mutated lactonase as defined above, said composition being applied for the prevention and/or treatment of plant infections such as fire blight, blackleg, rots, cankers, wilting, necrosis, broussin disease, Stewart's disease, Granville's disease, Moko's disease, yellow vine disease.

In this particular embodiment, said infections are caused by bacteria using homoserine lactone substrates to communicate.

In this particular embodiment, said bacteria are in particular selected from: Acidithiobacillus sp., Agrobacterium sp., Azospirillum sp., Bradyrhizobium sp., Burkholderia sp., Dickeya sp., Erwinia sp., Gluconacetobacter sp., Mesorhizobium sp., Nitrobacter sp., Pantoea sp., Pectobacterium sp., Pseudomonas sp., Ralstonia sp., Rhizobium sp., Serratia sp. and Sinorhizobium sp.

In another particular embodiment, the invention relates to the use of at least one mutated lactonase as defined above for the prevention and/or treatment of plant infections such as fire blight, blackleg, rots, cankers, wilt, necrosis, broussin disease, Stewart's disease, Granville's disease, Moko's disease, yellow vine disease.

In this particular embodiment, said infections are caused by bacteria using homoserine lactone substrates to communicate.

In this particular embodiment, said bacteria are in particular selected from: Acidithiobacillus sp., Agrobacterium sp., Azospirillum sp., Bradyrhizobium sp., Burkholderia sp., Dickeya sp., Erwinia sp., Gluconacetobacter sp., Mesorhizobium sp., Nitrobacter sp., Pantoea sp., Pectobacterium sp., Pseudomonas sp., Ralstonia sp., Rhizobium sp., Serratia sp. and Sinorhizobium sp.

In a particular embodiment, the invention relates to a composition comprising as active ingredient at least one mutated lactonase as defined above, for use on material contaminated or liable to be contaminated by bacteria using homoserine lactone substrates to communicate and form biofilms.

In a particular embodiment, said material contaminated or liable to be contaminated by bacteria using homoserine lactone substrates to communicate and form biofilms is selected from:
medical devices such as dressings, catheters, endoscopes, implants and nebulisers
medical equipment
submerged surfaces such as boat hulls, port or oil infrastructures that can be the target of biofouling or biocorrosion,
industrial installations such as cooling towers, air-conditioning systems, bioreactors, piping, nebulisers, foggers and ponds,
swimming pools, spas, balneotherapy equipment and ponds.

In a particular embodiment, said biofilms contain in particular one of the following species: Aliivibrio sp., Chromobacterium sp., Dinoroseobacter sp., Halomonas sp., Pseudomonas sp., Roseobacter sp. and Vibrio sp.

In all these aspects and in a particular embodiment, the said mutated lactonase of the invention is present at an effective dose, which depends on the nature of the bacteria to be eliminated.

In all these aspects and in a particular embodiment, the composition as described above may additionally comprise at least one antibiotic, or at least one biocide, or at least one disinfecting agent or at least one bacteriophage.

According to the invention, “antibiotic” means any agent capable of killing a bacterium or reducing, limiting or inhibiting its growth.

According to the present invention, antibiotics can be bactericidal antibiotics or bacteriostatic antibiotics. “Bactericidal antibiotics” means any agent capable of killing bacteria. “Bacteriostatic antibiotics” means any agent capable of reducing, limiting or inhibiting bacterial growth, without killing the bacteria.

According to the invention, a “disinfectant” is any substance applied to a non-living (inert) or living object (such as the skin) and capable of killing or inhibiting the growth of microorganisms present on the object. A disinfectant used on the body, i.e. applied to external body surfaces such as the skin, is called an “antiseptic”.

According to the invention, “biocide” means any substance or preparation intended to destroy, repel or render harmless harmful organisms, to prevent the action of harmful organisms or to combat them, by chemical or biological action. In other words, biocides are substances that act on or against harmful organisms.

According to the invention, a “bacteriophage” is any virus capable of infecting bacteria. Two types of bacteriophage can be distinguished:
lytic phages, which infect the bacteria, hijack its cellular machinery to reproduce and destroy the cell to release new phages
lysogenic or temperate phages, which insert their DNA into the bacterium in the form of a prophage.

According to the present invention, “bacteriophages which may or may not be naturally present in the environment” means bacteriophages naturally present in the environment as well as bacteriophages not present in the environment and added by a third party in order to eliminate bacteria.

In a particular embodiment, said antibiotic may be selected from the group consisting of: Amikacin, Amoxicillin, Amoxicillin/clavulanate, Ampicillin, Amprolium, Apramycin, Aspoxicillin, Aureomycin, Avilamycin, Azithromycin, Bacitracin, Bambermycin, Baquiloprim, Benzylpenicillin, Bicozamycin, Carbadox, Cefacetrile, Cefalexin, Cefalonium, Cefalotin, Cefapyrin, Cefazoline, Cefdinir, Ceftazidime Cefquinome, Ceftiofur, Ceftriaxone, Cefuroxime, Chloramphenicol, Chlortetracycline, Ciprofloxacin, Clarithromycin, Clindamycin, Cloxacillin, Colistin, Dalbavancin, Danofloxacin, Decoquinate, Diclazuril, Dicloxacillin, Difloxacin, Doripenem, Doxycycline, Enramycin, Enrofloxacin, Ertapenem, Erythromycin, Florfenicol, Flumequine, Fosfomycin, Framycetin, Fusidic acid, Gentamicin, Gentamicin Sulphate, Gramicidin, Halofuginone hydrobromide, Hetacillin, Imipeneme, Imipenem/cilastatin, Josamycin, Kanamycin, Kitasamycin, Laidlomycin, Lasalocid, Levofloxacin, Lincomycin, Lincomycin hydrochloride, Maduramycin, Marbofloxacin, Mecillinam, Meropeneme, Miloxacin, Minocycline, Mirosamycin, Monensin, Moxifloxacin, Nafcillin, Nalidixic acid, Narasin, Neomycin, Neomycin/oxytetracycline, Neosporin, Nicarbazine, Norfloxacin, Novobiocin, Ofloxacin, Orbifloxacin, Oritavancin, Oxacillin, oxolinic acid, Oxytetracycline, Paromomycin, penethamate hydroxide, Penicillin, Penicillin G potassium, Penicillin procaine, Penicillin V potassium, Phenethicillin, Phenoxymethylpenicillin, Pirlimycin, Polymyxin, Polymyxin B, Polysporin (bacitracin/polymyxin), Pristinamycin, Rifampicin, Rifaximin, Roxarsone, Salinomycin, Semduramicin, Spectinomycin, Spiramycin, Streptomycin, Sulfachlorpyridazine, Sulfadiazine, Sulfadimerazine, Sulfadimethoxazole, Sulfadimethoxine and ormetoprim 5:3, Sulfadimidine, Sulfadoxine, Sulfafurazole, Sulfaguanidine, Sulfamethazine, Sulfamethoxazole/trimethoprim, Sulfamethoxine, Sulfamethoxypyridazine, Sulfamonomethoxine, Sulfanilamide, Sulfaquinoxaline, Sulfasalazine, Sulfisoxazole, Surfactin, Telavancin, Terdecamycin, Tetracycline, Thiamphenicol, Tiamulin, Ticarcillin, Tilmicosin, Tobicillin, Tobramycin, Trimethoprim, Trimethoprim/Sulfonamide, Tulathromycin, Tylosin, Valnemulin, Vancomycin, Virginiamycin.

In a particular embodiment, said biocide may be selected from the group consisting of: biocidally active peroxides such as hydrogen peroxide, mono- and polyfunctional alcohols, aldehydes, acids, ozone, naphtha compounds and compounds containing an alkali metal, a transition metal, a Group III or Group IV metal, a sulphur, a nitrogen or a halogen atom and mixtures of two or more thereof.

In a particular embodiment, said biocide is selected from the group consisting of: formaldehyde, glutaraldehyde, peracetic acid, alkali metal hypochlorites, quaternary ammonium compounds, 2-amino-2-methyl-1-propanol, cetyltrimethylammonium bromide, cetylpyridinium chloride, 2,4,4-trichloro-2-hydroxy diphenyl ether, 1-(4-chlorophenyl)-3-(3,4-dichlorophenyl) urea, zinc oxide, zinc ricinoleate, pentachlorophenol, copper naphthenate, tributyltin oxide, dichlorophene, p-nitrophenol, p-chloro-m-xylenol, beta-naphthol, 2,3,5,6-tetrachloro-4-(methylsulfonyl) pyridine, salicylanilide, bromoacetic acid, alkyl quaternary ammonium acetate, sodium ethyl mercury thiosalicylate, sodium orthophenylphenate, n-alkyl (C2 to Cs) dimethyl benzyl ammonium chloride, organoborates, 2,2-(1-methyltrimethylene dioxy)-bis-(4-methyl-1,3,2-dioxaborinane), 2,2-oxybis(4,4,6-trimethyl)-1,3,2-dioxaborinane, ethylene glycol monomethyl ether, parahydroxybenzoates, organic boron compounds, 8-hydroxyquinoline, sodium pentachlorophenate, alkyl dimethyl ethyl benzyl ammonium chloride, alkylammonium salts, 1,3,5-triethylhexahydro-1,3,5-triazine, strontium chromate, halogenated phenols, 2-bromo-4-phenylphenol, silver salts such as silver nitrate, silver chloride, silver oxide and elemental silver, organic peroxides, silver sulphadiazine, sodium dichloro-S-triazinetrione, 4-chloro-2-cyclohexylphenol, 2-chloro-4-nitrophenol, paraffin substitutes, 3-chloro-3-nitro-2-butanol, 2-chloro-2-nitro-1-butanol stearate, 2-chloro-2-nitrobutyl acetate, 4-chloro-4-nitro-3-hexanol, 1-chloro-1-nitro-1-propanol, 2-chloro-2-nitro-1-propanol, triethyltin chloride, 2,4,5-trichlorophenol, 2,4,6-trichlorophenol, 1,3-dichloro-5,5-dimethylhydantoin, tris(hydoxymethyl)nitromethane, nitroparaffins, 2-nitro-2-ethyl-1,3-propanediol, 2-ethyl-2-nitro-1,3-propanediol, 2-methyl-2-nitro-1,3-propanediol, hexahydro-1,3,5-tris(2-hydroxyethyl)-S-triazine, hexahydro-1,3,5-tris(tetrahydro-2-furanyl)-methyl-S-triazine, methylene bis(thiocyanate), 2,2-dibromo-3-nitrilopropionamide, Beta-bromo-3-nitrostyrene, fluorinated compounds, N-ethyl-N-methyl-4-(trifluoromethyl)-2-(3,4-dimethoxyphenyl) benzamide, pentachlorophenol, dichlorophene, orthophenylphenol, di-bicyclo (3,1,1 or 2,2,1)-heptyl polyamines, di-bicyclo-(3,1,1 or 2,2,1)-heptanyl polyamines, zinc, bromine isothiazolinone.

In a particular embodiment, said disinfecting agent may comprise an alcohol, chlorine, aldehyde, oxidising agent, iodine, ozone, phenolic compound, quaternary ammonium compound or a mixture of two or more thereof.

In a particular embodiment, said disinfecting agent may comprise formaldehyde, orthophthalaldehyde, glutaraldehyde, silver dihydrogen citrate, polyaminopropyl biguanide, sodium bicarbonate, lactic acid, chlorine bleach, methanol, ethanol, n-propanol, 1-propanol, 2-propanol, isopropanol, hypochlorite, chlorine dioxide, dichloroisocyanurate, monochloroisocyanurate, hydantoin, sodium hypochlorite, calcium hypochlorite, sodium dichloroisocyanurate, sodium chlorite, 4-methylbenzenesulphonamide, sodium salt, 2,4-dichlorobenzyl alcohol, performic acid, paracetic acid, potassium permanganate, potassium peroxymonosulphate, phenol, phenylphenol, chloroxylenol, hexachlorophene, thymol, amylmetacresol, benzalkonuim chloride, cetyltrimethylammonium bromide, cetylpyridinium chloride, benzethonium chloride, boric acid, brilliant green, chlorhexidine gluconate, iodised providone, mercurochrome, manuka honey, octenidine dihydrochloride, polyhexamethylene biguanide, Peru balsam, hydrogen peroxide, organic peroxide, peroxyacid, organic hydroperoxide, peroxide salt, acid peroxides.

In a particular embodiment, the said bacteriophage may belong to the family of Myoviridae, Siphoviridae, Podoviridae, Corticoviridae, Cystoviridae, Inoviridae, Leviviridae, Microviridae, Plasmaviridae and Tectiviridae.

In another aspect, the invention relates to a method of preventing and/or treating pathologies linked to bacterial infections, comprising the administration of a mutated lactonase as defined above.

In another embodiment, the invention concerns a method of preventing and/or treating pathologies linked to bacterial infections, comprising the administration of a mutated lactonase belonging to the family of hyperthermophilic phosphotriesterase-like lactonases, as defined above.

Thus, in all these embodiments, said mutated lactonase may be any of the mutated lactonases described in any of the embodiments described in the first aspect of the invention.

In another embodiment, said bacterial infections may be bacterial infections in plants such as fire blight, blackleg, rots, cankers, wilting, necrosis, broussin disease, Stewart's disease, Granville's disease, Moko's disease, yellow vine disease.

In another embodiment, said bacterial infections may be bacterial infections in animals such as dysbiosis.

In another embodiment, said bacterial infections may be human bacterial infections such as pneumonia, nosocomial diseases, wounds, burns, eye infections, diabetic foot, dysbiosis or dental plaque.

### Examples

### Materials and Methods

### Substrates

The synthetic homoserine lactones (C4-HSL, C6-HSL, 3-oxo-C6-HSL, C8-HSL, 3-oxo-C10-HSL and 3-oxo-C12-HSL) were all purchased from COGER.

### Preparation of Media and Cultures

Chromobacterium violaceum CV026 and C. violaceum 12742 were grown in Luria-Bertani (LB) medium. P. putida KS35 was grown in LB medium supplemented with 50 μg/mL kanamycin. V. harveyi DSM623 was grown in AB medium (0.3 M NaCI, 0.05 M MgSO₄, 0.2% casamino acids (Difco), supplemented with 200 μL 1M potassium phosphate (pH 7.0), 200 μL 0.1 M L-arginine, and 250 μL 80% Glycerol for a final volume of 20 mL). Serratia sp. 39006 was grown in PGM medium (plant Peptone 5 g/L (Sigma) and 1% Glycerol). All bacteria were grown at 30° C.

### Production-Purification of Wild-Type and Variant SsoPox

The genes encoding the SsoPox variants were cloned into a pET22b plasmid. Production was carried out using Escherichia coli strain BL21(DE3)-pGro7/GroEL. Cultures were grown in self-inducible ZYP medium (supplemented with 100 μg·mL⁻¹ ampicillin and 34 μg·mL⁻¹ chloramphenicol). When the 600 nm absorbance reached a value of 0.8-1, CoCl₂ was added (final concentration 0.2 mM) along with L-arabinose (final concentration 2 g·L⁻¹) to induce the production of GroEL/ES chaperones and the temperature was lowered to 23° C. for 20 hours. Cells were harvested by centrifugation (4400 g, 10° C., 20 min) and resuspended in lysis buffer (50 mM HEPES pH 8, 150 mM NaCl, 0.2 mM CoCl₂, 0.25 mg·mL⁻¹ lysozyme, 0.1 mM PMSF and 10 μg·mL⁻¹ DNAseI) and stored at −80° C. Cells were thawed and lysed by three 30-second sonication steps (Qsonica, Q700; Amplitude 45). Cell debris was removed by centrifugation (20,000 g, 10° C., 15 min). As SsoPox and its variants are hyperthermostable, a pre-purification step was carried out by heating the lysate for 30 minutes at 70° C. Precipitated host proteins were removed by centrifugation (20,000 g, 10° C., 15 minutes). SsoPox and its variants were collected by precipitation with ammonium sulphate (75%) and resuspended in 8 ml SsoPox buffer (50 mM HEPES pH 8, 150 mM NaCl). The remaining ammonium sulphate was removed by injection onto a desalting column (HiPrep 26/10 desalting, GE Healthcare; ÄKTA Avant) and concentrated to 2 mL for separation by size exclusion chromatography (HiLoad 16/600 Superdex™ 75 μg, GE Healthcare; ÄKTA Avant). Final purity was checked by SDS-PAGE and protein concentration was measured using the Bradford protocol(1).

### Mutagenesis

Saturation mutagenesis directed at residues 266, 270, 271, 272, 273, 274 and 275 of the gene encoding SsoPox V82I was performed using degenerate NNS primers and the plasmid pET22b carrying the gene encoding SsoPox V82I as template. The following primers were used for mutagenesis:

A266NNS direct strand:
(SEQ ID NO: 9)
5′-GATTGGGGCACCNNSAAACCGGAATATA-3′
A266NNS reverse strand:
(SEQ ID NO: 10)
5′-CTAACCCCGTGGNNSTTTGGCCTTATAT-3′
Y270NNS direct strand:
(SEQ ID NO: 11)
5′-CGCAAAACCGGAANNSAAACCGAAAC-3′
Y270NNS reverse strand:
(SEQ ID NO: 12)
5′-GTTTCGGTTTSNNTTCCGGTTTTGCG-3′
K271NNS direct strand:
(SEQ ID NO: 13)
5′-CAAAACCGGAATATNNSCCGAAACTGGC-3′
K271NNS reverse strand:
(SEQ ID NO: 14)
5′-GCCAGTTTCGGSNNATATTCCGGTTTTG-3′
P272NNS direct strand:
(SEQ ID NO: 15)
5′-CCGGAATATAAANNSAAACTGGCACCG-3′
P272 reverse strand:
(SEQ ID NO: 16)
5′-CGGTGCCAGTTTSNNTTTATATTCCGG-3′
K273NNS direct strand:
(SEQ ID NO: 17)
5′-GGAATATAAACCGNNSCTGGCACCGCGT-3′
K273NNS reverse strand:
(SEQ ID NO: 18)
5′-ACGCGGTGCCAGSNNCGGTTTATATTCC-3′
L274NNS direct strand:
(SEQ ID NO: 19)
5′-TATAAACCGAAANNSGCACCGCGTTG-3′
L274NNS reverse strand:
(SEQ ID NO: 20)
5′-CAACGCGGTGCSNNTTTCGGTTTATA-3′
A275G-L274NNS direct strand:
(SEQ ID NO: 21)
5′-TATAAACCGAAANNSGGGCCGCGTTG-3′
A275G-L274NNS reverse strand:
(SEQ ID NO: 22)
5′-CAACGCGGCCCSNNTTTCGGTTTATA-3′
A275NNS direct strand:
(SEQ ID NO: 23)
5′-AAACCGAAACTGNNSCCGCGTTGGAG-3′
A275NNS reverse strand:
(SEQ ID NO: 24)
5′-TTTGGCTTTGACNNSGGCGCAACCTC-3′

PCR amplifications were performed with 2.5 U of PfuTurbo DNA polymerase (Agilent) according to the manufacturer's recommendations [95° C., 5 min; 20× (95° C., 30 s; 55° C., 1 min; 68° C., 15 min); 68° C., 25 min]. DNA was digested with the enzyme DpnI to remove the methylated parental template. Competent E. coli BL21(DE3)-pGro7/GroEL cells were transformed with the plasmid mixture and plated on LB agar supplemented with 100 μg·mL⁻¹ ampicillin and 34 μg·mL⁻¹ chloramphenicol. From a theoretical diversity of 20 sequences per residue, 88 variants per residue were collected and cultured in a microplate containing LB (100 μg·mL⁻¹ ampicillin and 34 μg·mL⁻¹ chloramphenicol) and 16% Glycerol. Residues 263 to 279 were alanine-scanned (i.e. replaced in alanines by all other possible amines) and the A266X-A275X double mutants were synthesised using GenScript.

The screening methods are described below. The plasmids corresponding to the most interesting variants were extracted and the genes encoding the SsoPox variants were sequenced.

### Library Screening

The plasmid library was used to transform E. coli BL21(DE3)-pGro7/GroEL to obtain colonies with mutated SsoPox genes. Randomly selected clones (88) were grown in a 96-well plate in 1 mL of ZYP medium. Chaperone production was induced after 5 h of culture at 37° C. by reducing the temperature to 23° C., adding CoCl₂ (0.2 mM) and arabinose (0.2%, w/v). After 20 h of growth, enzyme lysates were obtained by partial purification of the protein (heating at 70° C. for 30 min), followed by centrifugation (3000 rpm, 20 min). The screening test consisted of mixing a few microlitres of enzyme lysate (5 to 20 μL) with different concentrations of C4-HSL, C6-HSL, 3-oxo-C6-HSL, 3-oxo-C8, 3-oxo-C10 and 3-oxo-C12 ranging from 5 μM to 1 mM in LB medium. The rapportice strain CV026 or P. putida KS35 was then inoculated to the thousandth, grown overnight and the production of violacein for CV026 or fluorescence for P. putida KS35 was measured.

### Measurement of Lactonase Activity

The kinetic parameters of the lactonase activity were obtained using a protocol previously described in Hiblot et al. (2012)(2). The hydrolysis over time of lactones was monitored in lactonase buffer (2.5 mM bicine pH 8.3, 150 mM NaCl, 0.2 mM CoCl₂, 0.25 mM cresol violet and 0.5% DMSO) over a concentration range of 0 to 4 mM depending on the solubilities of the different lactones. Cresol violet (pKa 8.3 at 25° C.) is a pH indicator used to monitor hydrolysis of the lactone ring by acidification of the medium. The molar extinction coefficient at 577 nm was evaluated by measuring the absorbance of the buffer over a range of acetic acid concentrations from 0 to 0.35 mM. For all experiments, each time point was run in triplicate and Gen5.1 software was used to evaluate the initial degradation rate at each substrate concentration. Kinetic parameters were obtained using a regression of the Michaelis-Menten equation with GraphPad Prism 7 software.

### Thermostability

Melting temperatures (Tm) were obtained by differential scanning fluorimetry (DSF). Experiments were performed on the CFX96 Touch™ real-time PCR detection system (Bio-Rad). SsoPox variants were diluted to 0.2 mg·mL⁻¹ in Tris buffer (50 mM Tris-HCl, pH 7) supplemented with SYPRO® orange 200× (Sigma-Aldrich). Denaturation was monitored using the FRET channel. The temperature was increased from 35 to 95° C. (in increments of 0.5° C./15 sec). For some variants, guanidinium chloride was used at concentrations between 0.5 and 2 M. The data were fitted with Boltzmann's sigmoidal equation using GraphPad Prism 7 software, and the Tm at 0 M guanidinium chloride was extrapolated by linear regression.

### Results

### 1. V82I Mutation

During the first rounds of mutagenesis, a spontaneous mutation appeared at position 82, replacing the initial Valine with an Isoleucine. This mutation, although positioned far from the active site or loop 8, significantly improved the activities of SsoPox lactonase for 5 of the 6 substrates tested (Table 2).

Protein sequence: V82I
(SEQI ID NO: 25)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKLAPRWSITLIFEDTIPFLKRNG
VNEEVIATIFKENPKKFFS

TABLE 2
Kinetic parameters measured for the SsoPox V82I
mutant and improvement compared with SsoPox WT.

| | | k_{cat}/K_{M} | |
|---|---|---|---|
| Substrate | SsoPox | (M⁻¹ · s⁻¹) | Improvement/wt |
| C4-HSL | wt° | 11.62 | 1 |
| | | (±0.72) | |
| | V82I | 1.02 | 8.5 |
| | | (±0.17) × 10² | |
| C6-HSL | wt° | 7.84 | 1 |
| | | (±0.87) × 10² | |
| | V82I | 2.15 | 2.8 |
| | | (±0.24) × 10³ | |
| 3-oxo- | wt° | 6.87 | 1 |
| C6-HSL | | (±2.20) × 10¹ | |
| | V82I | 2.35 | 5.6 |
| | | (±0.25) × 10² | |
| C8-HSL | wt° | 6.90 | 1 |
| | | (±1.27) × 10³ | |
| | V82I | 9.21 | 3.4 |
| | | (±0.53) × 10³ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |

These results highlight the importance of position V82 in SsoPox activity. The V82I mutation was then added to the W263I variant, showing an overall increase in lactonase activity on each substrate tested (Table 3). This mutation was then retained on the template gene for subsequent rounds of mutagenesis.

Protein sequence: V821/W263I
(SEQI ID NO: 46)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDIGTAKPEYKPKLAPRWSITLIFEDTIPFLKRNGV
NEEVIATIFKENPKKFFS

TABLE 3
Kinetic parameters measured for the SsoPox V82I/W263I
mutant and improvement compared with SsoPox W263I.

| | | k_{cat}/K_{M} | |
|---|---|---|---|
| Substrate | SsoPox | (M⁻¹ · s⁻¹) | Improvement/wt |
| C4-HSL | wt° | 11.62 | 1 |
| | | (±0.72) | |
| | W263I | 3.61 | 3.1 |
| | | (±3.93) × 10¹ | |
| | V82I/W263I | 8.40 | 7.2 |
| | | (±4.52) × 10¹ | |
| C6-HSL | wt° | 7.84 | 1 |
| | | (±0.87) × 10² | |
| | W263I | 4.58 | 0.6 |
| | | (±0.48) × 10² | |
| | V82I/W263I | 5.95 | 0.8 |
| | | (±0.68) × 10² | |
| 3-oxo- | wt° | 6.87 | 1 |
| C6-HSL | | (±2.20) × 10¹ | |
| | W263I | 4.34 | 0.6 |
| | | (±1.79) × 10¹ | |
| | V82I/W263I | 8.88 | 1.3 |
| | | (±3.61) × 10¹ | |
| C8-HSL | wt° | 6.90 | 1 |
| | | (±1.27) × 10³ | |
| | W263I | 5.75 | 0.8 |
| | | (±0.9) × 10³ | |
| | V82I/W263I | 7.22 | 1.05 |
| | | (±1.2) × 10³ | |
| 3-oxo- | wt° | 3.16 | 1 |
| C10-HSL | | (±0.33) × 10⁴ | |
| | W263I | 8.50 | 0.03 |
| | | (±1.36) × 10² | |
| | V82I/W263I | 1.44 | 0.05 |
| | | (±0.17) × 10³ | |
| 3-oxo- | wt° | 2.22 | 1 |
| C12-HSL | | (±0.68) × 10³ | |
| | W263I | 1.89 | 8.5 |
| | | (±0.45) × 10⁴ | |
| | V82I/W263I | 2.82 | 12.7 |
| | | (±0.68) × 10⁴ | |

### 2. Alanine-Scanning

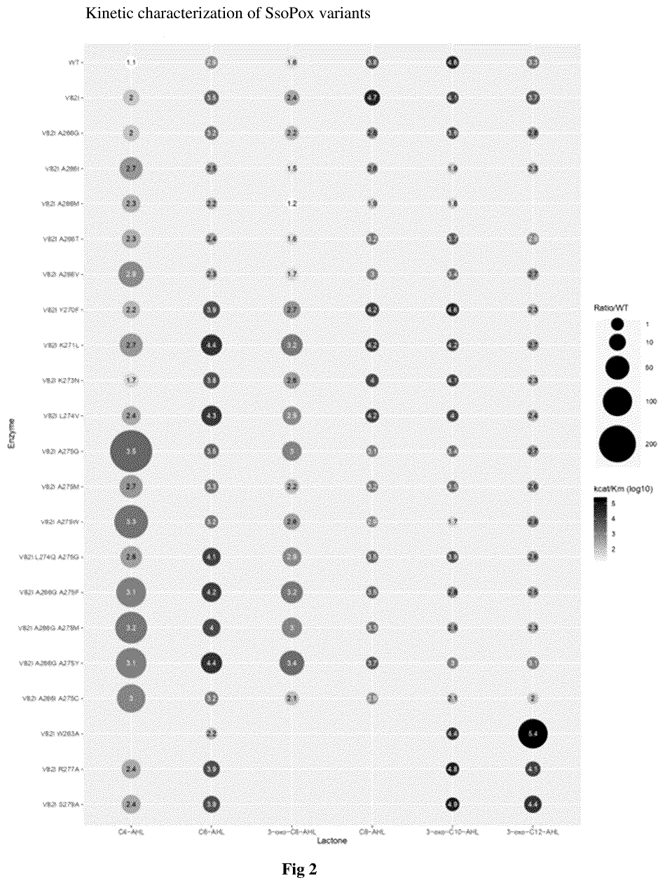

All the amino acids in loop 8 (with the exception of the two alanine residues: A266 and A275) were mutated by an alanine (alanine-scanning) to probe their respective impact on lactonase activity. The 15 enzymes obtained were as follows: W263A, G264A, T265A, K267A, P268A, E269A, Y270A, K271A, P272A, K273A, L274A, P276A, R277A, W278A, S279A, and all possess the V82I mutation. The activity of the variants was screened on five acyl-homoserine lactones (C4-HSL, C6-HSL, 3-oxo-C8-HSL, 3-oxo-C10-HSL, 3-oxo-C12-HSL) with different acyl chain lengths to characterise variations in enzymatic activities (FIG. 2).

5 of the 14 mutations in loop 8 residues increased the relative activity on C4-HSL compared with the reference (FIG. 2-A). 12 of the 14 mutations improved the degradation of C6-HSL (FIG. 2-B). For 3-oxo-C8-HSL, mutations of 13 residues had a positive impact on degradation (FIG. 2-C). 10 mutations on loop 8 increased the activity on 3-oxo-C10-HSL (FIG. 2-D) while 3 mutations significantly increased the activity on 3-oxo-C12-HSL (FIG. 2-E).

These results confirm the impact of mutations in SsoPox loop 8 residues on lactonase activity. These impacts highlight the possibility of modulating SsoPox activity by mutating loop 8 residues.

Following these results, residues A266, Y270, K271, P272, K273, L274 and A275 of loop 8 were exhaustively mutated by all other amino acids by site-directed saturation mutagenesis. The resulting enzyme libraries were screened on HSL short chains to identify variants with improved activity.

### 3. Kinetic Characterisation of Mutants

### a. V82I/W263A

Detected as an improved variant on 3-oxo-C10 and 3-oxo-C12-HSL, SsoPox V82I/W263A was purified and characterised (Table 4). This mutant had significantly increased activity on 3-oxo-C12-HSL, with a kcat/K_{M} 101-fold higher than SsoPox WT.

Protein sequence: V82I/W263A
(SEQ ID NO: 26)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDAGTAKPEYKPKLAPRWSITLIFEDTIPFLKRNGV
NEEVIATIFKENPKKFFS

TABLE 4
Kinetic parameters measured for the SsoPox V82I/W263A
mutant and improvements compared with SsoPox WT.

| | | k_{cat}/K_{M} | |
|---|---|---|---|
| Substrate | SsoPox | (M⁻¹ · s⁻¹) | Improvement/wt |
| C4-HSL | wt° | 11.62 | 1 |
| | | (±0.72) | |
| | V82I | 1.02 | 8.5 |
| | | (±0.17) × 10² | |
| | V82I/W263A | ND | — |
| C6-HSL | wt° | 7.84 | 1 |
| | | (±0.87) × 10² | |
| | V82I | 2.15 | 2.8 |
| | | (±0.24) × 10³ | |
| | V82I/W263A | 1.66 | 0.2 |
| | | (±0.97) × 10² | |
| 3-oxo- | wt° | 4.39 | 1 |
| C8-HSL | | (±0.8) × 10³ | |
| | V82I | 1.39 | 0.3 |
| | | (±0.19) × 10³ | |
| | V82I/W263A | 9.98 | 0.22 |
| | | (±1.78) × 10² | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/W263A | 2.45 | 0.77 |
| | | (±0.32) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/W263A | 2.24 | 101 |
| | | (±0.48) × 10⁵ | |

### b. A266NNS Enzyme Bank

Five variants were isolated from the A266NNS library by CV026 screening for short-chain AHLs. The corresponding mutations were identified as follows:

Protein sequences:
-V82I/A266G
(SEQ ID NO: 27)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTGKPEYKPKLAPRWSITLIFEDTIPFLKRNG
VNEEVIATIFKENPKKFFS
-V82I/A266T
(SEQ ID NO: 28)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTTKPEYKPKLAPRWSITLIFEDTIPFLKRNGV
NEEVIATIFKENPKKFFS
-V82I/A266V
(SEQ ID NO: 29)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTVKPEYKPKLAPRWSITLIFEDTIPFLKRNG
VNEEVIATIFKENPKKFFS
-V82I/A266I
(SEQ ID NO: 30)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTIKPEYKPKLAPRWSITLIFEDTIPFLKRNGV
NEEVIATIFKENPKKFFS
-V82I/A266M
(SEQ ID NO: 31)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTMKPEYKPKLAPRWSITLIFEDTIPFLKRNG
VNEEVIATIFKENPKKFFS

The catalytic efficiencies (kcat/KM) of these variants were determined on six HSLs (C4-HSL, C6-HSL, 3-oxo-C6-HSL, C8-HSL, 3-oxo-C10-HSL, 3-oxo-C12-HSL) (Table 5).

TABLE 5
Kinetic parameters measured for SsoPox V82I/A266
mutants and improvements compared with SsoPox WT.

| | | k_{cat}/K_{M} | |
|---|---|---|---|
| Substrate | SsoPox | (M⁻¹ · s⁻¹) | Improvement/wt |
| C4-HSL | wt° | 11.62 | 1 |
| | | (±0.72) | |
| | V82I | 1.02 | 8.5 |
| | | (±0.17) × 10² | |
| | V82I/A266G | 1.08 | 9 |
| | | (±0.31) × 10² | |
| | V82I/A266I | 5.22 | 45 |
| | | (±1.05) × 10² | |
| | V82I/A266M | 1.97 | 17 |
| | | (±0.6) × 10² | |
| | V82I/A266T | 2.20 | 19 |
| | | (±0.65) × 10² | |
| | V82I/A266V | 7.57 | 65 |
| | | (±2.54) × 10² | |
| C6-HSL | wt° | 7.84 | 1 |
| | | (±0.87) × 10² | |
| | V82I | 2.15 | 2.8 |
| | | (±0.24) × 10³ | |
| | V82I/A266G | 1.66 | 2.1 |
| | | (±0.21) × 10³ | |
| | V82I/A266I | 3.21 | 0.4 |
| | | (±0.65) × 10² | |
| | V82I/A266M | 1.64 | 0.2 |
| | | (±0.39) × 10² | |
| | V82I/A266T | 2.33 | 0.3 |
| | | (±0.39) × 10² | |
| | V82I/A266V | 2.11 | 0.3 |
| | | (±0.65) × 10² | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/A266G | 1.52 | 2.2 |
| | | (±0.15) × 10² | |
| | V82I/A266I | 3.27 | 0.5 |
| | | (±1.19) × 10¹ | |
| | V82I/A266M | 1.50 | 0.2 |
| | | (±1.14) × 10¹ | |
| | V82I/A266T | 4.05 | 0.6 |
| | | (±2.13) × 10¹ | |
| | V82I/A266V | 5.14 | 0.7 |
| | | (±1.69) × 10¹ | |
| C8-HSL | wt° | 6.90 | 1 |
| | | (±1.27) × 10³ | |
| | V82I | 2.37 | 3.4 |
| | | (±0.56) × 10⁴ | |
| | V82I/A266G | 6.23 | 0.09 |
| | | (±1.1) × 10² | |
| | V82I/A266I | 4.41 | 0.06 |
| | | (±1.57) × 10² | |
| | V82I/A266M | 8.10 | 0.01 |
| | | (±2.23) × 10¹ | |
| | V82I/A266T | 1.71 | 0.25 |
| | | (±0.32) × 10³ | |
| | V82I/A266V | 9.92 | 0.14 |
| | | (±1.66) × 10² | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/A266G | 8.47 | 0.268 |
| | | (±1.39) × 10³ | |
| | V82I/A266I | 8.39 | 0.003 |
| | | (±2.72) × 10¹ | |
| | V82I/A266M | 6.02 | 0.002 |
| | | (±2.16) × 10¹ | |
| | V82I/A266T | 4.73 | 0.150 |
| | | (±0.93) × 10³ | |
| | V82I/A266V | 2.57 | 0.081 |
| | | (±0.38) × 10³ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/A266G | 6.56 | 0.30 |
| | | (±3.01) × 10² | |
| | V82I/A266I | 2.16 | 0.10 |
| | | (±0.48) × 10² | |
| | V82I/A266M | ND | ND |
| | V82I/A266T | 7.29 | 0.33 |
| | | (±2.17) × 10² | |
| | V82I/A266V | 5.60 | 0.25 |
| | | (±2.05) × 10² | |

The 5 mutants, screened on short-chain HSLs, showed increased activity on C4-HSL, with SsoPox V82I/A266V up to 65 times more active on C4-HSL. SsoPox V82I/A266G had 2-fold enhanced activity on C6-HSL and 3-oxo-C6-HSL. For longer-chain HSLs, all mutants showed reduced activity compared with the wild-type enzyme (Table 5).

### c. Y270NNS Enzyme Bank

The Y270NNS enzyme library was used to identify a variant of interest: V82I/Y270F.

Protein sequence:
(SEQ ID NO: 32)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEFKPKLAPRWSITLIFEDTIPFLKRNGV
NEEVIATIFKENPKKFFS

The catalytic efficiencies (kcat/K_{M}) of this variant were determined for six HSLs (C4-HSL, C6-HSL, 3-oxo-C6-HSL, C8-HSL, 3-oxo-C10-HSL, 3-oxo-C12-HSL).

TABLE 6
Kinetic parameters measured for SsoPox V82I/Y270F
and improvements compared with SsoPox WT.

| | | k_{cat}/K_{M} | |
|---|---|---|---|
| Substrate | SsoPox | (M⁻¹ · s⁻¹) | Improvement/wt |
| C4-HSL | wt° | 11.62 | 1 |
| | | (±0.72) | |
| | V82I | 1.02 | 8.5 |
| | | (±0.17) × 10² | |
| | V82I/Y270F | 1.55 × 10² | 13 |
| C6-HSL | wt° | 7.84 | 1 |
| | | (±0.87) × 10² | |
| | V82I | 2.15 | 2.8 |
| | | (±0.24) × 10³ | |
| | V82I/Y270F | 8.12 | 10 |
| | | (±1.09) × 10³ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/Y270F | 4.93 | 12 |
| | | (±0.75) × 10² | |
| C8-HSL | wt° | 6.90 | 1 |
| | | (±1.27) × 10³ | |
| | V82I | 2.37 | 3.4 |
| | | (±0.56) × 10⁴ | |
| | V82I/Y270F | 1.62 | 2.35 |
| | | (±0.16) × 10⁴ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/Y270F | 4.22 | 1.17 |
| | | (±0.51) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/Y270F | 2.21 × 10² | 0.1 |

As observed with the A266NNS enzyme library, SsoPox V82I/Y270F has improved activities on C4-HSL, C6-HSL and 3-oxo-C6 HSL. However, the decrease in activity on C8-HSL and 3-oxo-C10-HSL was less drastic compared with the wild-type enzyme (Table 6).

### d. K271NNS Enzyme Bank

The K271NNS enzyme library led to the identification of the V82I/K271L variant.

Protein sequence:
(SEQ ID NO: 33)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYLPKLAPRWSITLIFEDTIPFLKRNGV
NEEVIATIFKENPKKFFS

The catalytic efficiencies (kcat/K_{M}) of this variant were determined on six HSLs (C4-HSL, C6-HSL, 3-oxo-C6-HSL, C8-HSL, 3-oxo-C10-HSL, 3-oxo-C12-HSL).

TABLE 7
Kinetic parameters measured for SsoPox V82I/K271L
and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/K271L | 5.31 x 10² | 44 |
| C6-HSL | wt° | 7.84 | 1 |
| | | (±0.87) × 10² | |
| | V82I | 2.15 | 2.8 |
| | | (±0.24) × 10³ | |
| | V82I/K271L | 2.39 | 31 |
| | | (±0.17) × 10⁴ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/K271L | 1.43 | 34 |
| | | (±0.2) × 10³ | |
| C8-HSL | wt° | 6.90 | 1 |
| | | (±1.27) × 10³ | |
| | V82I | 2.37 | 3.4 |
| | | (±0.56) × 10⁴ | |
| | V82I/K271L | 1.46 | 2.12 |
| | | (±0.38) × 10⁴ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/K271L | 1.44 | 0.4 |
| | | (±0.46) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/K271L | 4.69 | 0.21 |
| | | (±0.79) × 10² | |

The activity of SsoPox V82I/K271L on short-chain HSLs was greatly enhanced, with a 44-fold increase on C4-HSLs (Table 7).

### e. P272NNS Enzyme Bank

Screening of the P272NNS enzyme library revealed the V82I/P272L mutant with improved activity on 3-oxo-C8-HSL.

Protein sequence: V82I/P272L
(SEQI ID NO: 47)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKLKLAPRWSITLIFEDTIPFLKRNG
VNEEVIATIFKENPKKFFS

### f. K273NNS Enzyme Bank

The K273NNS enzyme library led to the identification of the V82I/K273N variant.

Protein sequence:
(SEQ ID NO: 34)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPNLAPRWSITLIFEDTIPFLKRNG
VNEEVIATIFKENPKKFFS

The catalytic efficiencies (kcat/K_{M}) of these variants were determined on six HSLs (C4-HSL, C6-HSL, 3-oxo-C6-HSL, C8-HSL, 3-oxo-C10-HSL, 3-oxo-C12-HSL).

TABLE 8
Kinetic parameters measured for SsoPox V82I/K273N
and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/K273N | 5.34 × 10¹ | 4 |
| C6-HSL | wt° | 7.84 | 1 |
| | | (±0.87) × 10² | |
| | V82I | 2.15 | 2.8 |
| | | (±0.24) × 10³ | |
| | V82I/K273N | 6.82 | 9 |
| | | (±0.82) × 10³ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/K273N | 4.08 | 10 |
| | | (±0.4) × 10² | |
| C8-HSL | wt° | 6.90 | 1 |
| | | (±1.27) × 10³ | |
| | V82I | 2.37 | 3.4 |
| | | (±0.56) × 10⁴ | |
| | V82I/K273N | 9.89 | 1.43 |
| | | (±0.36) × 10³ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/K273N | 1.16 | 0.32 |
| | | (±0.31) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/K273N | 1.84 × 10² | 0.08 |

The K273N mutation improved the activities for C6-HSL and 3-oxo-C6-HSL (Table 8).

### g. L274NNS Enzyme Bank

The L274NNS enzyme library led to the identification of the V82I/L274V variant.

Protein sequence:
(SEQ ID NO: 35)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKVAPRWSITLIFEDTIPFLKRNG
VNEEVIATIFKENPKKFFS

The catalytic efficiencies (kcat/K_{M}) of these variants were determined on six HSLs (C4-HSL, C6-HSL, 3-oxo-C6-HSL, C8-HSL, 3-oxo-C10-HSL, 3-oxo-C12-HSL).

TABLE 9
Kinetic parameters measured for SsoPox V82I/L274V
and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/L274V | 2.26 × 10² | 19 |
| C6-HSL | wt° | 7.84 | 1 |
| | | (±0.87) × 10² | |
| | V82I | 2.15 | 2.8 |
| | | (±0.24) × 10³ | |
| | V82I/L274V | 2.04 | 26 |
| | | (±0.14) × 10⁴ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/L274V | 7.22 | 17 |
| | | (±1.05) × 10² | |
| C8-HSL | wt° | 6.90 | 1 |
| | | (±1.27) × 10³ | |
| | V82I | 9.21 | 3.4 |
| | | (±0.53) × 10³ | |
| | V82I/L274V | 9.89 | 2.25 |
| | | (±0.36) × 10³ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/L274V | 9.21 | 0.26 |
| | | (±2.61) × 10³ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/L274V | 2.66 | 0.12 |
| | | (±3.33) × 10² | |

The L274V mutation improved activity on C4-HSL, C6-HSL and 3-oxo-C6-HSL (Table 9).

### h. A275NNS Enzyme Bank

Screening of the A275NNS library led to the identification of three variants. The corresponding mutations were identified as follows:

Protein sequences:
-V82I/A275G
(SEQ ID NO: 36)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKLGPRWSITLIFEDTIPFLKRNG
VNEEVIATIFKENPKKFFS
-V82I/A275M
(SEQ ID NO: 37)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKLMPRWSITLIFEDTIPFLKRNG
VNEEVIATIFKENPKKFFS
-V82I/A275W
(SEQ ID NO: 38)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKLWPRWSITLIFEDTIPFLKRNG
VNEEVIATIFKENPKKFFS

The catalytic efficiencies (kcat/K_{M}) of these variants were determined for six HSLs (C4-HSL, C6-HSL, 3-oxo-C6-HSL, C8-HSL, 3-oxo-C10-HSL, 3-oxo-C12-HSL).

TABLE 10
Kinetic parameters measured for SsoPox V82I/A266
mutants and improvements compared with SsoPox WT.

| | | k_{cat}/K_{M} | |
|---|---|---|---|
| Substrates | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/A275G | 3.45 | 297 |
| | | (±0.51) × 10³ | |
| | V82I/A275M | 5.29 | 46 |
| | | (±1.07) × 10² | |
| | V82I/A275W | 1.85 | 159 |
| | | (±0.17) × 10³ | |
| C6-HSL | wt° | 7.84 | 1 |
| | | (±0.87) × 10² | |
| | V82I | 2.15 | 2.8 |
| | | (±0.24) × 10³ | |
| | V82I/A275G | 1.98 | 2.5 |
| | | (±0.27) × 10³ | |
| | V82I/A275M | 2.02 | 2.6 |
| | | (±0.21) × 10³ | |
| | V82I/A275W | 1.48 | 1.9 |
| | | (±0.18) × 10³ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/A275G | 9.02 | 13.1 |
| | | (±1.08) × 10² | |
| | V82I/A275M | 1.66 | 2.4 |
| | | (±0.16) × 10² | |
| | V82I/A275W | 4.31 | 6.3 |
| | | (±0.52) × 10² | |
| C8-HSL | wt° | 6.90 | 1 |
| | | (±1.27) × 10³ | |
| | V82I | 9.21 | 3.4 |
| | | (±0.53) × 10³ | |
| | V82I/A275G | 1.27 | 0.18 |
| | | (±0.25) × 10³ | |
| | V82I/A275M | 1.54 | 0.22 |
| | | (±0.35) × 10³ | |
| | V82I/A275W | 7.10 | 0.10 |
| | | (±1.07) × 10² | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/A275G | 2.61 | 0.083 |
| | | (±0.47) × 10³ | |
| | V82I/A275M | 3.04 | 0.096 |
| | | (±0.61) × 10³ | |
| | V82I/A275W | 4.56 | 0.001 |
| | | (±1.93) × 10¹ | |
| | V82I/A266I/A275C | 1.26 | 0.004 |
| | | (±0.61) × 102 | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/A275G | 4.90 | 0.22 |
| | | (±0.96) × 10² | |
| | V82I/A275M | 3.90 | 0.18 |
| | | (±1.18) × 10² | |
| | V82I/A275W | 6.01 | 0.27 |
| | | (±2.21) × 10² | |

Mutations in residue A275 were used to identify the most interesting mutants for C4-HSL degradation: mutants V82I/A275G, V82I/A275M and V92I/A275W were 297, 46 and 159 times more active than SsoPox WT on this lactone. An improvement was also noted for C6-HSL and 3-oxo-C6-HSL. Activity on longer-chain lactones decreased compared with the wild-type enzyme (Table 10).

### i. V82I/R277A

Detected as an improved variant on all the lactones tested, SsoPox V82I/R277A was purified and characterised (Table 11). This mutant was 1.8 to 20.4 times more active than the wild-type enzyme on the various lactones tested.

Protein sequence: V82I/R277A
(SEQ ID NO: 39)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKLAPAWSITLIFEDTIPFLKRNG
VNEEVIATIFKENPKKFFS

TABLE 11
Kinetic parameters measured for the SsoPox V82I/R277A
mutant and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/R277A | 2.37 | 20.4 |
| | | (±2.74) × 10² | |
| C6-HSL | wt° | 7.84 | 1 |
| | | (±0.87) × 10² | |
| | V82I | 2.15 | 2.8 |
| | | (±0.24) × 10³ | |
| | V82I/R277A | 7.64 | 9.6 |
| | | (±2.39) × 10³ | |
| 3-oxo- | wt° | 4.39 | 1 |
| C8-HSL | | (±0.8) × 10³ | |
| | V82I | 1.39 | 0.3 |
| | | (±0.19) × 10³ | |
| | V82I/R277A | 1.22 | 2.8 |
| | | (±0.3) × 10⁴ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/R277A | 5.66 | 1.8 |
| | | (±0.65) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/R277A | 1.18 | 5.3 |
| | | (±0.14) × 10⁴ | |

### j. V82I/S279A

Detected as an improved variant on all the lactones tested, SsoPox V82I/S279A was purified and characterised (Table 12). This mutant increased its activity by up to 19.6-fold on C4-HSL.

Protein sequence: V82I/S279A
(SEQ ID NO: 40)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKLAPRWAITLIFEDTIPFLKRNG
VNEEVIATIFKENPKKFFS

TABLE 12
Kinetic parameters measured for the SsoPox V82I/S279A
mutant and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/S279A | 2.28 | 19.6 |
| | | (±2.11) × 10² | |
| C6-HSL | wt° | 7.84 | 1 |
| | | (±0.87) × 10² | |
| | V82I | 2.15 | 2.8 |
| | | (±0.24) × 10³ | |
| | V82I/S279A | 8.52 | 10.8 |
| | | (±2.16) × 10² | |
| 3-oxo- | wt° | 4.39 | 1 |
| C8-HSL | | (±0.8) × 10³ | |
| | V82I | 1.39 | 0.3 |
| | | (±0.19) × 10³ | |
| | V82I/S279A | 1.78 | 4 |
| | | (±0.41) × 10⁴ | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/S279A | 8.46 | 2.3 |
| | | (±1.47) × 10⁴ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/S279A | 2.76 | 12.4 |
| | | (±0.8) × 10⁴ | |

### k. A266NNS-A275NNS Enzyme Bank

Mutations in residues A266 and A275 have led to the identification of SsoPox variants that are more active towards short-chain AHLs. A new enzyme library was created, combining random mutations in these two residues. Screening of the library led to the identification of four mutants with improved kinetic parameters.

Protein sequences:
-V82I/A266G/A275F
(SEQ ID NO: 41)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTGKPEYKPKLFPRWSITLIFEDTIPFLKRNGV
NEEVIATIFKENPKKFFS
-V82I/A266G/A275M
(SEQ ID NO: 42)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTGKPEYKPKLMPRWSITLIFEDTIPFLKRNG
VNEEVIATIFKENPKKFFS
-V82I/A266G/A275Y
(SEQ ID NO: 43)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTGKPEYKPKLYPRWSITLIFEDTIPFLKRNG
VNEEVIATIFKENPKKFFS
-V82I/A266I/A275C
(SEQ ID NO: 44)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTIKPEYKPKLCPRWSITLIFEDTIPFLKRNGV
NEEVIATIFKENPKKFFS

The catalytic efficiencies (kcat/K_{M}) of these variants were determined on six HSLs (C4-HSL, C6-HSL, 3-oxo-C6-HSL, C8-HSL, 3-oxo-C10-HSL, 3-oxo-C12-HSL).

TABLE 13
Kinetic parameters measured for SsoPox V82I/A266/A275
mutants and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/A266G/A275F | 1.27 × 10³ | 106 |
| | V82I/A266G/A275M | 1.56 × 10³ | 130 |
| | V82I/A266G/A275Y | 1.36 × 10³ | 113 |
| | V82I/A266I/A275C | 1.09 × 10³ | 91 |
| C6-HSL | wt° | 7.84 | 1 |
| | | (±0.87) × 10² | |
| | V82I | 2.15 | 2.8 |
| | | (±0.24) × 10³ | |
| | V82I/A266G/A275F | 1.73 | 22.0 |
| | | (±0.13) × 10⁴ | |
| | V82I/A266G/A275M | 1.10 | 14.0 |
| | | (±0.14) × 10⁴ | |
| | V82I/A266G/A275Y | 2.42 | 30.8 |
| | | (±0.3) × 10⁴ | |
| | V82I/A266I/A275C | 1.53 | 2.0 |
| | | (±0.17) × 10³ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/A266G/A275F | 1.52 | 22 |
| | | (±0.27) × 10³ | |
| | V82I/A266G/A275M | 1.08 | 16 |
| | | (±0.24) × 10³ | |
| | V82I/A266G/A275Y | 2.37 | 35 |
| | | (±0.39) × 10³ | |
| | V82I/A266I/A275C | 1.33 | 2 |
| | | (±0.32) × 10² | |
| C8-HSL | wt° | 6.90 | 1 |
| | | (±1.27) × 10³ | |
| | V82I | 9.21 | 3.4 |
| | | (±0.53) × 10³ | |
| | V82I/A266G/A275F | 3.19 | 0.46 |
| | | (±0.49) × 10³ | |
| | V82I/A266G/A275M | 1.87 | 0.27 |
| | | (±0.22) × 10³ | |
| | V82I/A266G/A275Y | 4.86 | 0.70 |
| | | (±0.96) × 10³ | |
| | V82I/A266I/A275C | 8.28 | 0.12 |
| | | (±0.91) × 10² | |
| 3-oxo- | wt° | 3.16 | 1 |
| C10-HSL | | (±0.33) × 10⁴ | |
| | V82I | 1.24 | 0.4 |
| | | (±0.2) × 10⁴ | |
| | V82I/A266G/A275F | 6.73 | 0.021 |
| | | (±1.86) × 10² | |
| | V82I/A266G/A275M | 3.34 | 0.011 |
| | | (±1.18) × 10² | |
| | V82I/A266G/A275Y | 9.84 | 0.031 |
| | | (±1.96) × 10² | |
| | V82I/A266I/A275C | 1.26 | 0.004 |
| | | (±0.61) × 10² | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/A266G/A275F | 3.07 | 0.14 |
| | | (±1.03) × 10² | |
| | V82I/A266G/A275M | 1.88 | 0.08 |
| | | (±0.77) × 10² | |
| | V82I/A266G/A275Y | 1.25 | 0.56 |
| | | (±4.15) × 10³ | |
| | V82I/A266I/A275C | 1.01 | 0.05 |
| | | (±0.24) × 10² | |

These triple mutants have improved activities on C4-HSL, up to 130-fold for SsoPox V82I/A266G/A275M. Their C6-HSL and 3-oxo-C6 HSL activities were improved by up to 35-fold. The V82I/A266G/A275Y variant had the highest kcat/K_{M} ever measured in a SsoPox variant (Table 13).

### l. L274NNS-A275G Enzyme Bank

Preliminary analysis of the enzyme structure showed that the A275G mutation changes the orientation of residue L274, leading to new interactions with the HSL substrate. A new enzyme library was created combining the A275G mutation with random mutations on L274. A mutant of interest was discovered: V82I/L274Q/A275G.

Protein sequence:
(SEQ ID NO: 45)
MRIPLVGKDSIESKDIGFTLIHEHLRVFSEAVRQQWPHLYNEDEEFRNAVNEVKRAM
QFGVKTIVDPTVMGLGRDIRFMEKIVKATGINLVAGTGIYIYIDLPFYFLNRSIDEIADL
FIHDIKEGIQGTLNKAGFVKIAADEPGITKDVEKVIRAAAIANKETKVPIITHSNAHNNT
GLEQQRILTEEGVDPGKILIGHLGDTDNIDYIKKIADKGSFIGLDRYGLDLFLPVDKRN
ETTLRLIKDGYSDKIMISHDYCCTIDWGTAKPEYKPKQGPRWSITLIFEDTIPFLKRNG
VNEEVIATIFKENPKKFFS

The catalytic efficiencies (kcat/K_{M}) of this variant were determined on six HSLs (C4-HSL, C6-HSL, 3-oxo-C6-HSL, C8-HSL, 3-oxo-C10-HSL, 3-oxo-C12-HSL).

TABLE 14
Kinetic parameters measured for SsoPox V82I/L274Q/A275G
and improvements compared with SsoPox WT.

| | | k_{cat}/K_{M} | |
|---|---|---|---|
| Substrate | SsoPox | (M⁻¹ · s⁻¹) | improvement/wt |
| C4-HSL | wt° | 11.62 | 1 |
| | | (±0.72) | |
| | V82I | 1.02 | 8.5 |
| | | (±0.17) × 10² | |
| | V82I/L274Q/A275G | 4.05 × 10² | 34 |
| C6-HSL | wt° | 7.84 | 1 |
| | | (±0.87) × 10² | |
| | V82I | 2.15 | 2.8 |
| | | (±0.24) × 10³ | |
| | V82I/L274Q/A275G | 1.22 | 16 |
| | | (±0.09) × 10⁴ | |
| 3-oxo- | wt° | 6.87 | 1 |
| C6-HSL | | (±2.20) × 10¹ | |
| | V82I | 2.35 | 5.6 |
| | | (±0.25) × 10² | |
| | V82I/L274Q/A275G | 7.76 | 18 |
| | | (±1.83) × 10² | |
| C8-HSL | wt° | 6.90 | 1 |
| | | (±1.27) × 10³ | |
| | V82I | 9.21 | 3.4 |
| | | (±0.53) × 10³ | |
| | V82I/L274Q/A275G | 8.4 | 0.49 |
| | | (±0.04) × 10³ | |
| 3-oxo- | wt° | 3.16 | 1 |
| C10-HSL | | (±0.33) × 10⁴ | |
| | V82I | 2.91 | 0.8 |
| | | (±0.81) × 10⁴ | |
| | V82I/L274Q/A275G | 8.4 | 0.23 |
| | | (±2.27) × 10³ | |
| 3-oxo- | wt° | 2.22 | 1 |
| C12-HSL | | (±0.68) × 10³ | |
| | V82I | 5.01 | 2.3 |
| | | (±1.31) × 10³ | |
| | V82I/L274Q/A275G | 4.39 | 0.2 |
| | | (±0.78) × 10² | |

These mutations combined to improve the activity of the enzyme on C4-HSL, C6-HSL and 3-oxo-C6-HSL (Table 14).

### m. Summary

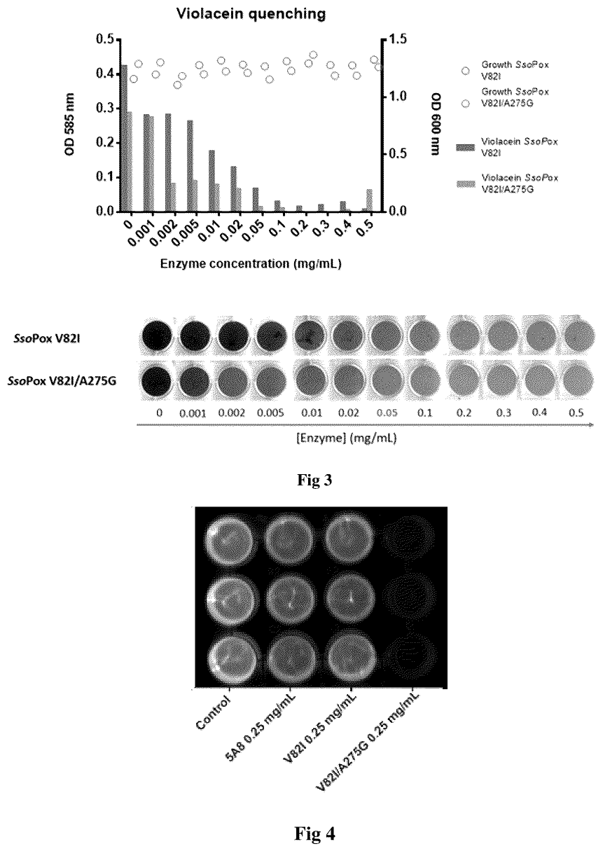

The SsoPox variants selected showed a strong increase in activity for short-chain HSLs, from C4-HSL to C6-HSL. The greatest improvements were obtained on C4-HSL, with several variants more than 100 times more active on this substrate. The kcat/K_{M} on C6-HSL also increased by a factor of 100 (FIG. 3).

In addition, all these variants retained a melting temperature above 85° C., thus not affecting the hyperthermostability of the enzyme (Table 15). As a result, it is possible to design variants combining several mutations without impacting the enzyme's ability to withstand potentially denaturing industrial transformation steps.

TABLE 15
Melting temperature of SsoPox variants.

| | Melting |
|---|---|
| | temperature |
| SsoPox | (° C.) |
| wt° | 106 |
| V82I | 96.5 |
| V82I/W263I | 90.3 |
| V82I/W263A | 83.5 |
| V82I/A266G | 89.6 |
| V82I/A266I | 92.8 |
| V82I/A266M | 98.6 |
| V82I/A266T | 89.3 |
| V82I/A266V | 91.7 |
| V82I/Y270F | 100 |
| V82I/K271L | 88 |
| V82I/K273N | 89 |
| V82I/L274V | 96 |
| V82I/A275G | 93.7 |
| V82I/A275M | 87.9 |
| V82I/A275W | 88.8 |
| V82I/R277A | 92.5 |
| V82I/A266G/A275F | 88.1 |
| V82I/A266G/A275M | 85.2 |
| V82I/A266G/A275Y | 87.1 |
| V82I/A266I/A275C | 93 |
| V82I/L274Q/A275G | 95 |

These results highlight the importance of loop 8 for SsoPox activity, and the possibility of modulating its spectrum of activity by mutating the residues making up this area of the enzyme.

### 4. Phenotypic Impact on Model Bacteria

The SsoPox mutants presented above have been shown to have a greater impact than the wild-type enzyme on quorum sensing phenotypes in several bacteria. The impact of the A275G mutation on quenching in various bacterial strains is described below.

a. Chromobacterium violaceum

Quorum sensing in Chromobacterium violaceum 12472 regulates the production of violacein, a violet pigment, through the production and detection of various HSLs. SsoPox is known to inhibit this production of violacein by degradation of HSLs, without interfering with bacterial growth. Here, the SsoPox V82I/A275G mutant is able to inhibit violacein production at lower concentrations than SsoPox V82I, confirming its enhanced degradation activity on short-chain HSLs (FIG. 4).

b. Vibrio harveyi

Vibrio harveyi quorum sensing induces several phenotypes, including the production of bioluminescence.

SsoPox V82I/A275G completely extinguishes the bioluminescence emission of V. harveyi, whereas the same concentration of SsoPox V82I does not reduce this phenotype compared with the inactive enzyme (FIG. 5).

c. Serratia sp. 39006

Serratia sp. ATCC 39006 is a Gram-negative bacterium that is virulent in potatoes and animal models. It produces two quorum sensing-regulated antibiotics, prodigiosin and a carbapenem(3). The impact of the SsoPox V82I/A275G mutant compared with SsoPox V82I on various phenotypes of this bacterial strain is being studied.

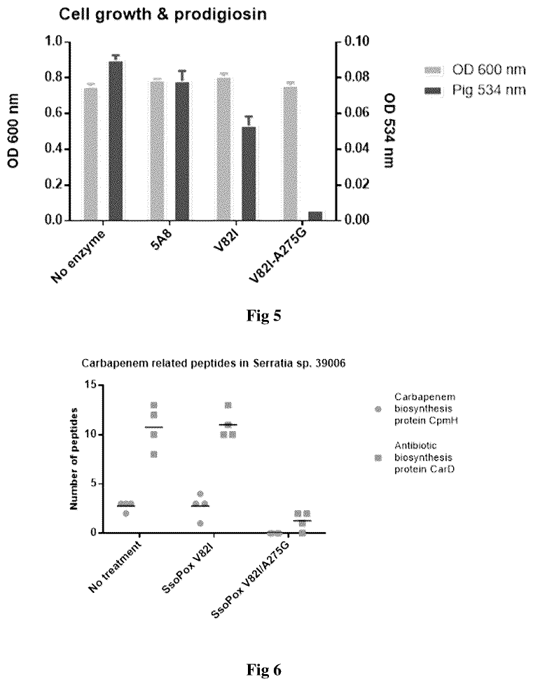

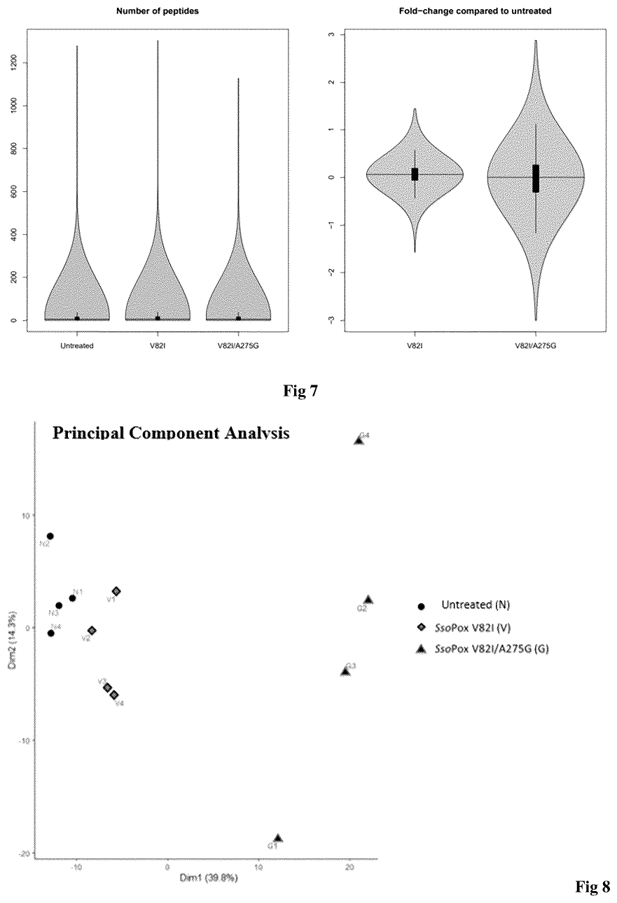

SsoPox V82I/A275G was able to completely inhibit prodigiosin production in Serratia sp. 39006, whereas SsoPox V82I only slightly reduced this phenotype compared to the control (FIG. 6). Similarly, treatment with SsoPox V82I/A275G significantly reduced the expression of carbapenem-related proteins, while treatment with SsoPox V82I showed no difference compared with the control (FIG. 7).

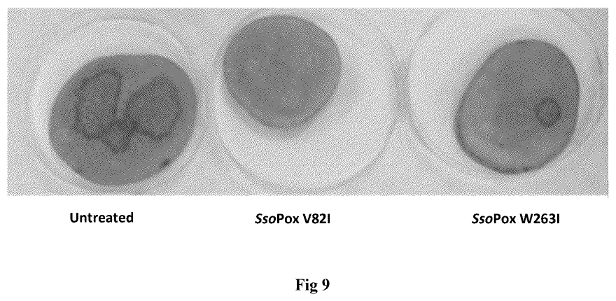

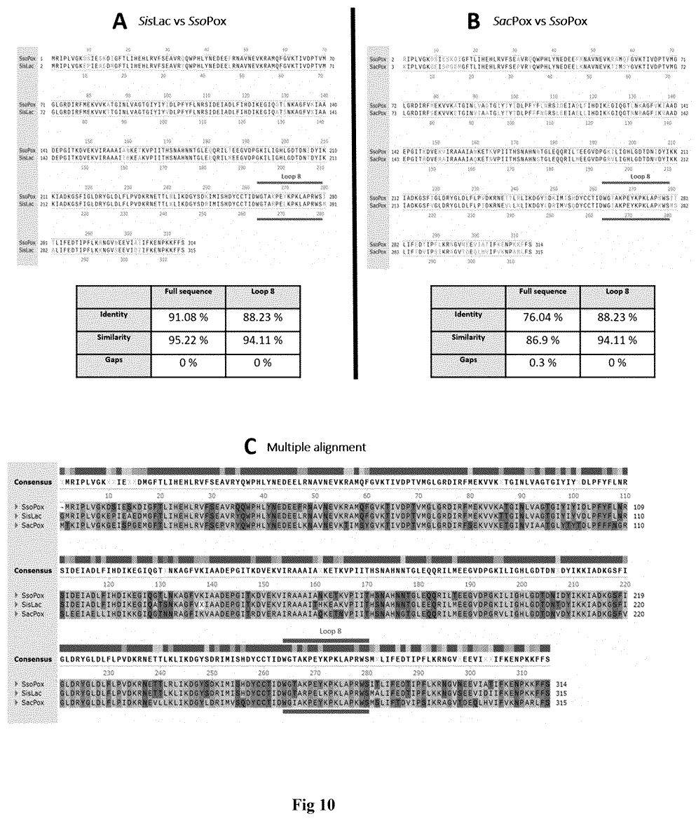

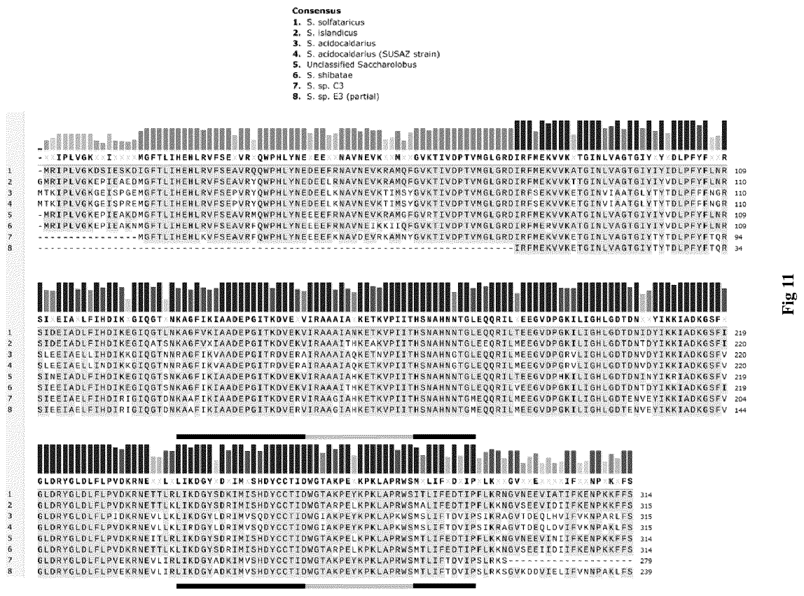

Proteomic analyses were performed on Serratia sp. 39006, grown without enzyme, with SsoPox V82I or SsoPox V82I/A275G. Treatment with SsoPox V82I/A275G resulted in greater changes in protein translation levels than SsoPox V82I compared with the untreated culture (FIG. 8-DB), despite an equivalent amount of peptides being detected (FIG. 8-A). These results confirm the greater influence of SsoPox V82I/A275G on Serratia phenotypes. Principal component analysis confirmed the difference in impact of these two enzymes on Serratia sp. 39006, with good separation of conditions on one dimension. (FIG. 9).

These results highlight the fact that the A275G mutation, which increases the degradation activity of short-chain HSLs in the SsoPox V82I variant, has a greater impact on QS-related phenotypes in three different bacteria, demonstrating the importance of loop 8 residues in improving the efficiency of quorum quenching by SsoPox.

d. Pectobacterium atrosepticum

Pectobacterium atrosepticum CFBP6276 was grown overnight at 30° C. Potato slices of the Mona Lisa variety, 5 mm thick, were prepared and pierced at three separate points. Each spot was filled with 20 μL of SsoPox buffer (control) or SsoPox buffer containing 0.1 mg of enzyme. After drying, each spot was inoculated with a drop of 20 μL SsoPox buffer containing 10³ CFU of P. atrosepticum CFBP6276 and incubated at room temperature (23-24° C.) and high humidity for 72 h.

No maceration was detected when inoculated potatoes were protected with the SsoPox V82I variant.

5. Sequence Similarity with Other Phosphotriesterase-Like-Lactonases (PLL)

Previous results demonstrate the impact of mutations in loop 8 residues on lactonase activity and the phenotypic impact of SsoPox. SsoPox have very similar protein sequences (FIG. 11) and three-dimensional structures(2, 4) with other PLLs from hyperthermophilic archaea: SisLac, isolated from Saccharolobus islandicus and SacPox from Saccharolobus acidocaldarius.

The conservation of loop 8 residues among these PLLs (88% identity and 94% similarity) confirms its essential role in the lactonase activity of PLLs.

All Saccharolobus LDPs share very high identity with the SsoPox sequence (>75%). Loop 8 is extremely conserved by each enzyme, with a minimum of 88% identity. The alignment of 49 additional amino acids adjacent to loop 8 returned a percentage identity of over 90%, with the exception of the S. acidocaldarius LRP with an identity of 79% (Table 16—FIG. 12).

TABLE 16
Percentage identity of various LRPs from the
Saccharolobus genus to the SsoPox sequence.
Alignments and identities were obtained using blastp.

| | complete | zone | |
|---|---|---|---|
| organism | sequence | L242-P289 | Loop 8 |
| S. islandicus | 91% | 90% | 88% |
| S. acidocaldarius | 76% | 79% | 88% |
| S. acidocaldarius | 76% | 77% | 88% |
| Unclassified | 92% | 94% | 94% |
| S. shibatae | 90% | 92% | 88% |
| S. sp. C3 | 84% | 90% | 100% |
| S. sp. E3 (partial) | 82% | 90% | 100% |

### REFERENCES

1. Bradford, M. M. (1976) A rapid and sensitive method for the quantitation of microgram quantities of protein utilizing the principle of protein-dye binding. Analytical Biochemistry. 72, 248-254
2. Hiblot, J., Gotthard, G., Chabriere, E., and Elias, M. (2012) Structural and Enzymatic characterization of the lactonase SisLac from Sulfolobus islandicus. PLoS ONE. 10.1371/journal.pone.0047028
3. Wilf, N. M., Williamson, N. R., Ramsay, J. P., Poulter, S., Bandyra, K. J., and Salmond, G. P. C. (2011) The RNA chaperone, Hfq, controls two luxR-type regulators and plays a key role in pathogenesis and production of antibiotics in Serratia sp. ATCC 39006: Role of Hfq in Serratia sp. ATCC 39006. Environmental Microbiology. 13, 2649-2666
4. Bzdrenga, J., Hiblot, J., Gotthard, G., Champion, C., Elias, M., and Chabriere, E. (2014) SacPox from the thermoacidophilic crenarchaeon Sulfolobus acidocaldarius is a proficient lactonase. BMC Res Notes. 7, 333

1.-11. (canceled)

12. A mutated lactonase belonging to the phosphotriesterase-like hyperthermophilic lactonase family, said mutated lactonase comprising:
a first mutation by substitution of an amino acid in a first consensus sequence SEQ ID NO: 1 of a wild-type lactonase,
which first consensus sequence in said wild-type lactonase is represented by SEQ ID NO: 1:
(SEQ ID: 1)
I-R-F-[M/S]-E-[K/R]-X-V-K-[A/T/E]-T-G-I-N
X represents the V amino acid,
which first consensus sequence in said mutated lactonase is represented by SEQ ID NO: 2:
(SEQ ID NO: 2)
I-R-F-[M/S]-E-[K/R]-X1-V-K-[A/T/E]-T-G-I-N
X₁ represents the substituted amino acid chosen from the group consisting of the hydrophobic amino acids V, I, L, M, F, G, A, P, W, Y and C, in particular A, G and I, in particular A or I,
at least one other mutation by substitution of an amino acid in a second consensus sequence of the wild-type lactonase represented by SEQ ID NO: 3:
(SEQ ID NO: 3)
Xa-G-[T/I]-Xb-[K/R]-P-E-Xc-Xd-Xe-Xf-Xg-Xh-P-Xi-
W-Xj,
Xa is selected from the group consisting of W, T, A, F, V, I, M and L,
Xb represents the A amino acid,
Xc is selected from the group consisting of Y and L,
Xd represents the K amino acid,
Xe represents the P amino acid,
Xf represents the K amino acid,
Xg represents the L amino acid,
Xh represents the A amino acid,
Xi is selected from the group consisting of R and K,
Xj represents the S amino acid,
which second consensus sequence in said substitution-mutated lactonase is represented by SEQ ID:4:
(SEQ ID NO: 4)
X₂-G-[T/I]-X₃-[K/R]-P-E-X₄-X₅-X₆-X₇-X₈-X₉-W-X₁₀-P-X₁₁
at least one of the amino acids X₂, X₃, X₄, X₅, X₆, X₇, X₈, X₉, X₁₀ or X₁₁ being substituted,
said mutated lactonase having increased lactonase activity compared with said wild-type lactonase.

13. The mutated lactonase according to claim 12, wherein,
the at least one other mutation by substitution concerns at least one of the amino acids X₂, X₃, X₄, X, X₅₆, X₇, X₈, X₉, X₁₀ or X₁₁ of the sequence SEQ ID NO: 4 in which:
X₂ is selected from the group consisting of the hydrophobic amino acids V, I, L, M, F, G, A, P, W, Y and C,
X₃ is selected from the group consisting of the hydrophobic amino acids V, I, L, M, F, G, P, W, Y and C,
X₄ is selected from the group consisting of the hydrophobic amino acids V, I, L, M, F, G, A, P, W, Y and C,
X₅ is selected from the group consisting of the hydrophobic amino acids V, I, L, M, F, G, A, P, W, Y and C,
X₆ is selected from the group consisting of the hydrophobic amino acids V, I, L, M, F, G, A, W, Y and C,
X₇ is selected from the group consisting of the polar amino acids S, T, N, Q, E, D, R and H,
X₈ is selected from the group consisting of the hydrophobic amino acids V, I, M, F, G, A, P, W, Y and C,
X₉ is selected from the group consisting of the hydrophobic amino acids V, I, L, M, F, G, P, W, Y and C,
X₁₀ is selected from the group consisting of the non-voluminous amino acids G, P, L, I, A, D, C, S, T and N, and
X₁₁ is selected from the group consisting of the hydrophobic amino acids V, I, L, M, F, G, A, P, W, Y and C.

14. The mutated lactonase according to claim 12, wherein X₁ represents I.

15. The mutated lactonase according to claim 12, wherein the at least one other mutation by substitution concerns at least one of the amino acids X₂, X₃, X₄, X₅, X₆, X₇, X₈, X₉, X₁₀ or X₁₁ of the sequence SEQ ID NO: 4 in which:
X₂ is selected from the group consisting of A and I,
X₃ is selected from the group consisting of V, I, M, G and T,
X₄ represents F,
X₅ represents L,
X₆ represents L,
X₇ represents N,
X₈ represents V,
X₉ is selected from the group consisting of F, M, G, Y, C and W,
X₁₀ represents A,
X₁₁ represents A.

16. The mutated lactonase according to claim 12, wherein the at least one other mutation by substitution concerns the amino acids X₃, and X₉, of the sequence SEQ ID NO: 4 in which:
X₃ is selected from the group consisting of I and G,
X₉ is selected from the group consisting of F, M, Y and C.

17. The mutated lactonase according to claim 12, in which the said mutated lactonase has a sequence identity of at least 85%, 90%, 95%, 96%, 97%, 98%, 99% or 100% with the sequences SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47,
provided that said mutated lactonase retains increased lactonase activity relative to said wild-type lactonase.

18. A method for disrupting the quorum-sensing of bacteria using homoserine lactone substrates to communicate, and for limiting or inhibiting the formation of biofilms comprising administering or using the mutated lactonase as defined in claim 12.

19. A method for the treatment of bacterial infections, such as pneumonia or nosocomial diseases, wounds, burns, ocular infections, diabetic foot, for the treatment of dysbiosis, or for the treatment of dental plaque, comprising administering to a patient in need thereof a composition comprising as active ingredient at least one mutated lactonase as defined according to claim 12,
said bacterial infections being preferably caused by bacteria using homoserine lactone substrates for communication, said bacteria being notably selected from: Acinetobacter sp., Brucella sp., Burkholderia sp., Chromobacterium sp., Enterobacter sp., Hafnia sp., Klebsiella sp., Kluyvera sp., Pandoraea sp., Proteus sp., Pseudomonas sp., Rahnella sp., Vibrio sp. and Yersinia sp.

20. A method for the treatment of bacterial infections and dysbiosis comprising administering to an animal in need thereof a composition comprising as active ingredient at least one mutated lactonase as defined according to claim 12,
said bacterial infections being preferably caused by bacteria using homoserine lactone substrates for communication, said bacteria being notably selected from: Aeromonas sp., Aliivibrio sp., Brucella sp., Burkholderia sp., Chromobacterium sp., Edwardsiella sp., Enterobacter sp., Halomonas sp., Pseudomonas sp., Vibrio sp. and Yersinia sp.

21. A method for the treatment of plant infections such as fire blight, blackleg, rots, cankers, wilting, necrosis, broussin disease, Stewart's disease, Granville's disease, Moko's disease and yellow vine disease comprising applying a phytosanitary composition comprising as active ingredient at least one mutated lactonase as defined according to claim 12,
said infections being preferably caused by bacteria using homoserine lactone substrates for communication, said bacteria being notably selected from: Acidithiobacillus sp., Agrobacterium sp., Azospirillum sp., Bradyrhizobium sp., Burkholderia sp., Dickeya sp., Erwinia sp., Gluconacetobacter sp., Mesorhizobium sp., Nitrobacter sp., Pantoea sp., Pectobacterium sp., Pseudomonas sp., Ralstonia sp., Rhizobium sp., Serratia sp. and Sinorhizobium sp.

22. A method for the treatment of material contaminated or liable to be contaminated by bacteria using homoserine lactone substrates to communicate and form biofilms comprising applying a composition comprising at least one mutated lactonase as defined according to claim 12, the said contaminated material being chosen from:
medical devices such as dressings, catheters, endoscopes, implants and nebulisers,
medical equipment,
submerged surfaces such as boat hulls, port or oil infrastructures that can be the target of biofouling or biocorrosion,
industrial installations such as cooling towers, air conditioning systems, bioreactors, piping, nebulisers, foggers and ponds, and
swimming pools, spas, balneotherapy equipment and ponds,
said biofilms preferably containing one of the following species: Aliivibrio sp., Chromobacterium sp., Dinoroseobacter sp., Halomonas sp., and Pseudomonas sp.


SEQUENCE LISTING (47 sequences)

SEQ ID NO: 1 - protein, 14 aa, synthetic construct
  REGION 1..14: note = Artificial sequence
  VAR_SEQ 4
  VAR_SEQ 6
  VAR_SEQ 7
  VAR_SEQ 10
IRFXEXXVKXTGIN

SEQ ID NO: 2 - protein, 14 aa, synthetic construct
  REGION 1..14: note = Artificial sequence
  VAR_SEQ 4
  VAR_SEQ 6
  VAR_SEQ 7
  VAR_SEQ 10
IRFXEXXVKXTGIN

SEQ ID NO: 3 - protein, 17 aa, synthetic construct
  REGION 1..17: note = Artificial sequence
  VAR_SEQ 1
  VAR_SEQ 3
  VAR_SEQ 4
  VAR_SEQ 5
  VAR_SEQ 8
  VAR_SEQ 9
  VAR_SEQ 10
  VAR_SEQ 11
  VAR_SEQ 12
  VAR_SEQ 13
  VAR_SEQ 15
  VAR_SEQ 17
XGXXXPEXXXXXXPXWX

SEQ ID NO: 4 - protein, 17 aa, synthetic construct
  REGION 1..17: note = Artificial sequence
  VAR_SEQ 1
  VAR_SEQ 3
  VAR_SEQ 4
  VAR_SEQ 5
  VAR_SEQ 8
  VAR_SEQ 9
  VAR_SEQ 10
  VAR_SEQ 11
  VAR_SEQ 12
  VAR_SEQ 13
  VAR_SEQ 15
  VAR_SEQ 17
XGXXXPEXXXXXXPXWX

SEQ ID NO: 5 - protein, 314 aa, Saccharolobus solfataricus
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 6 - protein, 315 aa, Sulfolobus acidocaldarius
MTKIPLVGKGEISPGEMGFTLIHEHLRVFSEPVRYQWPHLYNEDEELKNAVNEVKTIMSYGVKTIVDPTVMGLGRDIRFSEKVVKETGINVIAATGLYTYTDLPFFFNGRSLEEIAELLIHDIKKGIQGTNNRAGFIKVAADEPGITRDVERAIRAAAIAQKETNVPIITHSNAHNGTGLEQQRILMEEGVDPGRVLIGHLGDTDNVDYIKKIADKGSFVGLDRYGLDLFLPIDKRNEVLLKLIKDGYLDRIMVSQDYCCTIDWGIAKPEYKPKLAPKWSMSLIFTDVIPSIKRAGVTDEQLHVIFVKNPARLFS

SEQ ID NO: 7 - protein, 314 aa, Sulfolobus islandicus
MRIPLVGKEPIEAEDMGFTLIHEHLRVFSEAVRYQWPHLYNEDEELRNAVNEVKRAMQFGVKTIVDPTVMGLGRDIRFMEKVVKTTGINLVAGTGIYIYVDLPFYFLNRSIDEIADLFIHDIKEGIQATSNKAGFVKIAADEPGITKDVEKVIRAAAITHKEAKVPIITHSNAHNNTGLEEQRILMEEGVDPGKILIGHLGDTDNTDYIKKIADKGSFIGLDRYGLDLFLPVDKRNETTLKLIKDGYSDRIMISHDYCCTIDWGTARPELKPKLAPRWSMALIFEDTIPFLKKNGVSEEVIDIIFKENPKKFFS

SEQ ID NO: 8 - protein, 314 aa, Saccharolobus shibatae
MRIPLVGKEPIEAKNMGFTLIHEHLRVFSEAVRFQWPHLYNEEEEFRNAVNEIKKIIQFGVKTIVDPTVMGLGRDIRFMERVVKATGINLVAGTGIYIYVDLPFYFLNRSIEEIADLFIHDIKEGIQGTSNKAGFIKIAADEPGITKDVEKVIRAAAITHKETKVPIITHSNAHNNTGLEQQRILVEEGVDPGKILIGHLGDTDNTDYIKKIADKGSFIGLDRYGLDLFLPVDKRNETTLKLIKDGYSDRIMISHDYCCTIDWGTARPELKPKLAPRWSMTLIFEDTIPFLKKNGVSEEIIDIIFKENPKKFFS

SEQ ID NO: 9 - DNA, 28 bp, synthetic construct
  misc_feature 1..28: note = artificial sequence
  variation 13..14
  variation 15
  source 1..28: mol_type = other DNA
gattggggcaccnnsaaaccggaatata

SEQ ID NO: 10 - DNA, 28 bp, synthetic construct
  misc_feature 1..28: note = artificial sequence
  variation 13..14
  variation 15
  source 1..28: mol_type = other DNA
ctaaccccgtggnnstttggccttatat

SEQ ID NO: 11 - DNA, 26 bp, synthetic construct
  misc_feature 1..26: note = artificial sequence
  variation 14..15
  variation 16
  source 1..26: mol_type = other DNA
cgcaaaaccggaannsaaaccgaaac

SEQ ID NO: 12 - DNA, 26 bp, synthetic construct
  misc_feature 1..26: note = artificial sequence
  variation 11
  variation 12..13
  source 1..26: mol_type = other DNA
gtttcggtttsnnttccggttttgcg

SEQ ID NO: 13 - DNA, 28 bp, synthetic construct
  misc_feature 1..28: note = artificial sequence
  variation 15..16
  variation 17
  source 1..28: mol_type = other DNA
caaaaccggaatatnnsccgaaactggc

SEQ ID NO: 14 - DNA, 28 bp, synthetic construct
  misc_feature 1..28: note = artificial sequence
  variation 12
  variation 13..14
  source 1..28: mol_type = other DNA
gccagtttcggsnnatattccggttttg

SEQ ID NO: 15 - DNA, 27 bp, synthetic construct
  misc_feature 1..27: note = artificial sequence
  variation 13..14
  variation 15
  source 1..27: mol_type = other DNA
ccggaatataaannsaaactggcaccg

SEQ ID NO: 16 - DNA, 27 bp, synthetic construct
  misc_feature 1..27: note = artificial sequence
  variation 13
  variation 14..15
  source 1..27: mol_type = other DNA
cggtgccagtttsnntttatattccgg

SEQ ID NO: 17 - DNA, 28 bp, synthetic construct
  misc_feature 1..28: note = artificial sequence
  variation 14..15
  variation 16
  source 1..28: mol_type = other DNA
ggaatataaaccgnnsctggcaccgcgt

SEQ ID NO: 18 - DNA, 28 bp, synthetic construct
  misc_feature 1..28: note = artificial sequence
  variation 13
  variation 14..15
  source 1..28: mol_type = other DNA
acgcggtgccagsnncggtttatattcc

SEQ ID NO: 19 - DNA, 26 bp, synthetic construct
  misc_feature 1..26: note = artificial sequence
  variation 13..14
  variation 15
  source 1..26: mol_type = other DNA
tataaaccgaaannsgcaccgcgttg

SEQ ID NO: 20 - DNA, 26 bp, synthetic construct
  misc_feature 1..26: note = artificial sequence
  variation 12
  variation 13..14
  source 1..26: mol_type = other DNA
caacgcggtgcsnntttcggtttata

SEQ ID NO: 21 - DNA, 26 bp, synthetic construct
  misc_feature 1..26: note = artificial sequence
  variation 13..14
  variation 15
  source 1..26: mol_type = other DNA
tataaaccgaaannsgggccgcgttg

SEQ ID NO: 22 - DNA, 26 bp, synthetic construct
  misc_feature 1..26: note = artificial sequence
  variation 12
  variation 13..14
  source 1..26: mol_type = other DNA
caacgcggcccsnntttcggtttata

SEQ ID NO: 23 - DNA, 26 bp, synthetic construct
  misc_feature 1..26: note = artificial sequence
  variation 13..14
  variation 15
  source 1..26: mol_type = other DNA
aaaccgaaactgnnsccgcgttggag

SEQ ID NO: 24 - DNA, 26 bp, synthetic construct
  misc_feature 1..26: note = artificial sequence
  variation 13..14
  variation 15
  source 1..26: mol_type = other DNA
tttggctttgacnnsggcgcaacctc

SEQ ID NO: 25 - protein, 314 aa, synthetic construct
  source 1..314: note = mutant SEQ ID NO:5
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 26 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDAGTAKPEYKPKLAPRWSITLIFEDTIPFLKRNGVNEEVIATIFKENPKKFFS

SEQ ID NO: 27 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTGKPEYKPKLAPRWSITLIFEDTIPFLKRNGVNEEVIATIFKENPKKFFS

SEQ ID NO: 28 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTTKPEYKPKLAPRWSITLIFEDTIPFLKRNGVNEEVIATIFKENPKKFFS

SEQ ID NO: 29 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTVKPEYKPKLAPRWSITLIFEDTIPFLKRNGVNEEVIATIFKENPKKFFS

SEQ ID NO: 30 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTIKPEYKPKLAPRWSITLIFEDTIPFLKRNGVNEEVIATIFKENPKKFFS

SEQ ID NO: 31 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTMKPEYKPKLAPRWSITLIFEDTIPFLKRNGVNEEVIATIFKENPKKFFS

SEQ ID NO: 32 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEFKPKLAPRWSITLIFEDTIPFLKRNGVNEEVIATIFKENPKKFFS

SEQ ID NO: 33 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYLPKLAPRWSITLIFEDTIPFLKRNGVNEEVIATIFKENPKKFFS

SEQ ID NO: 34 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPNLAPRWSITLIFEDTIPFLKRNGVNEEVIATIFKENPKKFFS

SEQ ID NO: 35 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKVAPRWSITLIFEDTIPFLKRNGVNEEVIATIFKENPKKFFS

SEQ ID NO: 36 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLGPRWSITLIFEDTIPFLKRNGVNEEVIATIFKENPKKFFS

SEQ ID NO: 37 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLMPRWSITLIFEDTIPFLKRNGVNEEVIATIFKENPKKFFS

SEQ ID NO: 38 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLWPRWSITLIFEDTIPFLKRNGVNEEVIATIFKENPKKFFS

SEQ ID NO: 39 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPAWSITLIFEDTIPFLKRNGVNEEVIATIFKENPKKFFS

SEQ ID NO: 40 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWAITLIFEDTIPFLKRNGVNEEVIATIFKENPKKFFS

SEQ ID NO: 41 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTGKPEYKPKLFPRWSITLIFEDTIPFLKRNGVNEEVIATIFKENPKKFFS

SEQ ID NO: 42 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTGKPEYKPKLMPRWSITLIFEDTIPFLKRNGVNEEVIATIFKENPKKFFS

SEQ ID NO: 43 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTGKPEYKPKLYPRWSITLIFEDTIPFLKRNGVNEEVIATIFKENPKKFFS

SEQ ID NO: 44 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTIKPEYKPKLCPRWSITLIFEDTIPFLKRNGVNEEVIATIFKENPKKFFS

SEQ ID NO: 45 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKQGPRWSITLIFEDTIPFLKRNGVNEEVIATIFKENPKKFFS

SEQ ID NO: 46 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDIGTAKPEYKPKLAPRWSITLIFEDTIPFLKRNGVNEEVIATIFKENPKKFFS

SEQ ID NO: 47 - protein, 314 aa, synthetic construct
MRIPLVGKDSIESKDIGFTLIHEHLRVFSEAVRQQWPHLYNEDEEFRNAVNEVKRAMQFGVKTIVDPTVMGLGRDIRFMEKIVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKLKLAPRWSITLIFEDTIPFLKRNGVNEEVIATIFKENPKKFFS